(19) United States
(12) Patent Application Publication
(10) Pub. No.: US 2025/0090533 A1
(43) Pub. Date: Mar. 20, 2025

(54) COMBINATION THERAPY COMPRISING A MAT2A INHIBITOR AND A TAXANE

(71) Applicant: IDEAYA BIOSCIENCES, INC., South San Francisco, CA (US)

(72) Inventors: Claire L. Neilan, South San Francisco, CA (US); Marcus M. Fischer, South San Francisco, CA (US); Kimberline Yang Gerrick, South San Francisco, CA (US); Neil E. Bhola, South San Francisco, CA (US)

(21) Appl. No.: 18/708,487

(22) PCT Filed: Nov. 11, 2022
(86) PCT No.: PCT/US2022/079710

§ 371 Date: May 8, 2024

Related U.S. Application Data
(60) Provisional application No. 63/278,706, filed on Nov. 12, 2021.

(51) Int. Cl. A61K 31/519; A61K 31/337; A61P 35/00

(52) U.S. Cl. CPC A61K 31/519; A61K 31/337; A61P 35/00

(57) ABSTRACT

Provided herein is a combination of a methionine adenosyltransferase II alpha (MAT2A) inhibitor and a taxane, and methods of using such combinations to treat diseases or disorders associated with MAT2A, for example, cancer.

### RELATED APPLICATIONS

This application claims priority to U.S. Provisional Application No. 63/278,706, filed on Nov. 12, 2021, the entire content of which is hereby incorporated by reference in its entirety.

### BACKGROUND

Cancer is a leading cause of death throughout the world. A limitation of prevailing therapeutic approaches, e.g., chemotherapy and immunotherapy, is that their cytotoxic effects are not restricted to cancer cells and adverse side effects can occur within normal tissues.

Methionine adenosyltransferase 2A (MAT2A) is an enzyme that utilizes methionine (Met) and adenosine triphosphate (ATP) to generate s-adenosyl methionine (SAM). SAM is a primary methyl donor in cells used to methylate several substrates including DNA, RNA and proteins. One methylase that utilizes SAM as a methyl donor is protein arginine N-methyltransferase 5 (PRMT5). While SAM is required for PRMT5 activity, PRMT5 is competitively inhibited by 5′methylthioadenosine (MTA). Since MTA is part of the methionine salvage pathway, cellular MTA levels stay low in a process initiated by methylthioadenosine phosphorylase (MTAP).

MTAP is in a locus on chromosome 9 that is often deleted in cells of patients with cancers from several tissues of origin including central nervous system, pancreas, esophageal, bladder and lung (cBioPortal database). Loss of MTAP results in the accumulation of MTA making MTAP-deleted cells more dependent on SAM production, and thus MAT2A activity, compared to cells that express MTAP. In an shRNA cell-line screen across approximately 400 cancer cell lines, MAT2A knockdown resulted in the loss of viability in a larger percentage of MTAP-deleted cells compare to MTAP WT cells (see McDonald et. al. 2017 Cell 170, 577-592). Furthermore, inducible knockdown of MAT2A protein decreased tumor growth in vivo (see Marjon et. al., 2016 Cell Reports 15(3), 574-587). These results indicate that MAT2A inhibitors may provide a novel therapy for cancer patients including those with MTAP-deleted tumors.

Taxanes are a class of diterpenes that act as mitotic inhibitors. Their principal mechanism of action is the disruption of microtubule function. Microtubules are essential to cell division, and taxanes stabilize GDP-bound tubulin in the microtubule, thereby inhibiting the process of cell division as depolymerization is prevented.

Despite many recent advances in cancer therapies, there remains a need for more effective and/or enhanced treatment for those individuals suffering the effects of cancer.

### SUMMARY

Provided herein is a combination product comprising a methionine adenosyltransferase II alpha (MAT2A) inhibitor and a taxane. The combination product is useful for the treatment of a variety of cancers, including solid tumors. The combination product is also useful for the treatment of any number of MAT2A-associated and/or microtubule-associated diseases. Further, the combination product is useful for the treatment of a variety of diseases or disorders treatable by inhibiting MAT2A. In addition, the combination product is useful for treating MTAP-deficient tumors.

In an embodiment, provided herein is a combination of a methionine adenosyltransferase II alpha (MAT2A) inhibitor and a taxane.

In an embodiment, provided herein is a pharmaceutical composition comprising a therapeutically effective amount of a methionine adenosyltransferase II alpha (MAT2A) inhibitor and a second pharmaceutical composition comprising a therapeutically effective amount of a taxane.

In an embodiment, provided herein are methods of treating cancer in a subject in need thereof, the methods comprising administering to the subject a combination comprising a methionine adenosyltransferase II alpha (MAT2A) inhibitor and a taxane, thereby treating the cancer in the subject.

In an embodiment, provided herein are methods of treating cancer in a subject in need thereof, the methods comprising administering to the subject a combination comprising a MAT2A inhibitor and a taxane, together with at least a pharmaceutically acceptable carrier, thereby treating the cancer in the subject.

In still another embodiment, the cancer is characterized by a reduction or absence of methylthioadenosine phosphorylase (MTAP) gene expression, absence of MTAP gene, reduced function of MTAP protein, reduced level or absence of MTAP protein, MTA accumulation, or combination thereof.

In an embodiment, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a taxane, thereby treating the cancer in the subject.

In an embodiment, the MAT2A inhibitor is a compound of Formula I:

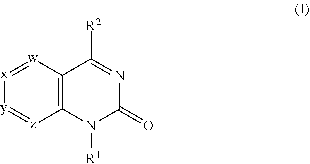

or a pharmaceutically acceptable salt thereof, wherein the variables of Formula I are defined below.

In another embodiment, the MAT2A inhibitor is Compound A having the following structural formula:

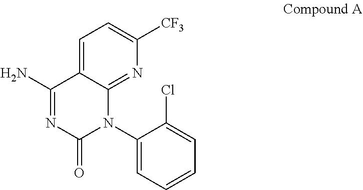 [SMILES: Nc1nc(=O)n(-c2ccccc2Cl)c2nc(C(F)(F)F)ccc12]

or a pharmaceutically acceptable salt thereof.

In another embodiment, the MAT2A inhibitor is Compound A1 having the following structural formula:

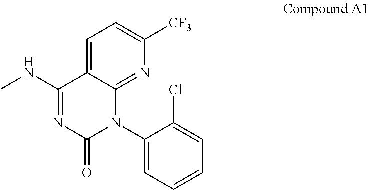 [SMILES: CNc1nc(=O)n(-c2ccccc2Cl)c2nc(C(F)(F)F)ccc12]

or a pharmaceutically acceptable salt thereof. Compound A1, and methods of making Compound A1 are disclosed in PCT/US19/65260 (WO 2020/123395).

MAT2A inhibitors for use in the combination therapy described herein are described in WO 2020/123395 (PCT/US19/65260). The generic and specific compounds described in these patent applications are incorporated herein by reference and can be used to treat cancer as described herein.

In yet another embodiment, the taxane is paclitaxel (referred herein as Compound B) having the following structural formula:

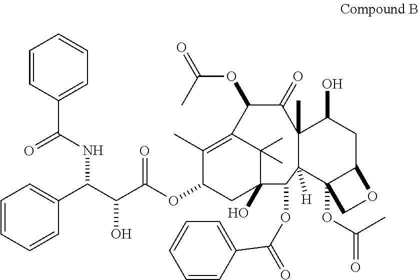

or a pharmaceutically acceptable salt thereof. The chemical name for paclitaxel is 5β,20-Epoxy-1,2α,4,7μ,10β,13α-hexahydroxytax-11-en-9-one 4,10-diacetate 2-benzoate 13-ester with (2R,3S)—N-benzoyl-3-phenylisoserine.

In still another embodiment, the taxane is protein-bound paclitaxel (also known as Abraxane®, and referred herein as Compound C). Compound C is also known as nab-paclitaxel (nanoparticle albumin-bound paclitaxel).

In another embodiment, the taxane is docetaxel (referred herein as Compound D):

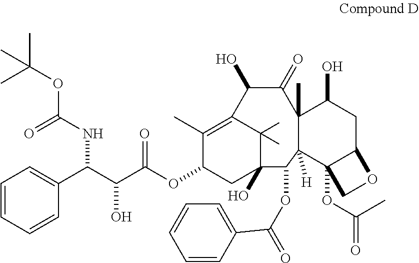

or a pharmaceutically acceptable salt thereof. The chemical name for docetaxel is (2R,3S)—N-carboxy-3-phenylisoserine, N-tert-butyl ester, 13-ester with 5β-20-epoxy-1,2α,4,7μ,10μ3,13α-hexahydroxytax-11-en-9-one 4-acetate 2-benzoate. In an embodiment, the Compound D is anhydrous. In an embodiment, the Compound D is a trihydrate.

### BRIEF DESCRIPTION OF THE DRAWINGS

FIG. 1 shows the efficacy of Compound A and Compound B in HCT-116 MTAP-deleted cells.

FIG. 2 shows the efficacy of Compound A and Compound B in HCT-116 WT cells.

FIG. 3 shows the efficacy of Compound A and Compound B in PAXF 2094.

FIG. 4 shows the efficacy of Compound A and Compound B in NCI-H838.

FIG. 5 shows the efficacy of Compound A and Compound B or Compound D in NCI-H838.

FIG. 6 shows the efficacy of Compound A and Compound B in NCI-H647.

FIG. 7 shows the efficacy of Compound A and Compound D in NCI-H647.

FIG. 8A, FIG. 8B, FIG. 8C, FIG. 8D, FIG. 8E, FIG. 8F, FIG. 8G, FIG. 8H, FIG. 8I, FIG. 8J, FIG. 8K, FIG. 8L, and FIG. 8M show the growth inhibition of Compound A and Compound B in 13 MTAP-deficient cell lines.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, FIG. 9L, and FIG. 9M show Loewe Synergy of Compound A and Compound B in 13 MTAP-deficient cell lines.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, FIG. 10L, and FIG. 10M show growth inhibition of Compound A and Compound D in 13 MTAP-deficient cell lines.

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L, and FIG. 11M show Loewe Synergy of Compound A and Compound D in 13 MTAP-deficient cell lines.

FIG. 12A, FIG. 12B, and FIG. 12C show growth inhibition of Compound A and Compound D in MTAPWT and MTAP-deficient cell lines.

FIG. 13A, FIG. 13B, FIG. 13C, FIG. 13D, FIG. 13E, FIG. 13F, FIG. 13G, FIG. 13H, and FIG. 13I show combination benefit of Compound A and Compound D in MTAPWT and MTAP-deficient cell lines.

### DETAILED DESCRIPTION

Provided herein is a combination therapy comprising a methionine adenosyltransferase II alpha (MAT2A) inhibitor, or a pharmaceutically acceptable salt thereof, and a taxane, or a pharmaceutically acceptable salt thereof. The combination therapy is useful for the treatment of a variety of cancers, including pancreatic cancer and lung cancer. In another aspect, the combination therapy is useful for the treatment of any number of MAT2A-associated diseases. In another aspect, the combination therapy is useful for the treatment of a disease or disorder treatable by inhibiting MAT2A.

Administering a combination of a methionine adenosyltransferase II alpha (MAT2A) inhibitor and a taxane can provide beneficial effects for treating cancer, e.g., solid tumors, in a subject. Such an approach—combination or co-administration of the two types of agents—may offer an uninterrupted treatment to an subject in need over a clinically relevant treatment period.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or +10%, including ±5%, +1%, and +0.1% from the specified value, as such variations are appropriate to perform the disclosed methods.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. In addition, the phrase “about ‘x’ to ‘y’” includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” as used herein, comprises the prevention of at least one symptom associated with or caused by the state, disease or disorder being prevented.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “single formulation” as used herein refers to a single carrier or vehicle formulated to deliver therapeutically effective amounts of both therapeutic agents to a patient. The single vehicle is designed to deliver a therapeutically effective amount of each of the agents, along with any pharmaceutically acceptable carriers or excipients. In some embodiments, the vehicle is a tablet, capsule, pill, or a patch. In other embodiments, the vehicle is a solution or a suspension.

As used herein “methionine adenosyltransferase II alpha inhibitor” or “MAT2A inhibitor” means an agent that modulates the activity of MAT2A or inhibits the production of S-adenosylmethionine (SAM) by methionine adenosyltransferase 2A (MAT2A).

As used herein, “taxane” refers to a diterpine having the following taxadiene core:

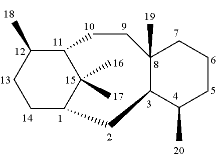

Examples of taxane include, but are not limited to, paclitaxel, docetaxel, nab-paclitaxel, or alternative formulations thereof.

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes a therapeutically effective amount of at least one of the agents along with pharmaceutically acceptable carriers and excipients. In some embodiments, the unit dose is one or more tablets, capsules, pills, or patches administered to the patient at the same time. The combination of agents described herein may display a synergistic effect.

The term “synergistic effect” as used herein, refers to action of two agents such as, for example, a methionine adenosyltransferase II alpha (MAT2A) inhibitor and a taxane, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., a methionine adenosyltransferase II alpha (MAT2A) inhibitor and a taxane, used together is greater than the sum of the effects that results from using the compounds separately.

In an embodiment, provided herein is a combination therapy comprising a therapeutically effective amount of a methionine adenosyltransferase II alpha (MAT2A) inhibitor and a taxane. A “therapeutically effective amount” of a combination of agents (i.e., a MAT2A inhibitor and a taxane) is an amount sufficient to provide an observable improvement over the baseline clinically observable signs and symptoms of the disorders treated with the combination.

An “oral dosage form” includes a unit dosage form prescribed or intended for oral administration.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond, e.g., propenyl, butenyl, and the like.

“Alkynyl” means a linear monovalent hydrocarbon radical of two to six carbon atoms or a branched monovalent hydrocarbon radical of three to six carbon atoms containing a triple bond, e.g., ethynyl, propynyl, butynyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with one alkoxy group, as defined above, e.g., 2-methoxyethyl, 1-, 2-, or 3-methoxypropyl, 2-ethoxyethyl, and the like.

“Alkoxyalkoxy” means a —OR radical where R is alkoxyalkyl as defined above e.g., methoxyethyloxy, ethyloxypropyloxy, and the like.

“Alkoxyalkylamino” means a —NRR′ radical where R is hydrogen or alkyl and R′ is alkoxyalkyl, each as defined above e.g., methoxyethylamino, methoxypropylamino, and the like.

“Alkylcarbonyl” means a —C(O)R radical where R is alkyl as defined herein, e.g., methylcarbonyl, ethylcarbonyl, and the like.

“Alkoxycarbonyl” means a —C(O)OR radical where R is alkyl as defined above, e.g., methoxycarbonyl, ethoxycarbonyl, and the like.

“Alkoxycarboxyalkyl” means an alkyl radical as defined above, that is substituted with an alkoxycarboxy group e.g., methylcarboxymethyl, ethylcarboxyethyl, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Alkylsulfonylalkyl” means a -(alkylene)-SO2R radical where R is alkyl as defined above, e.g., methylsulfonylethyl, ethylsulfonylmethyl, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen, alkyl, haloalkyl, hydroxyalkyl, alkoxyalkyl, or alkylcarbonyl, each as defined herein, e.g., aminomethyl, aminoethyl, methylaminomethyl, and the like.

“Aminoalkoxy” means a —OR radical where R is aminoalkyl as defined above e.g., aminoethyloxy, methylaminopropyloxy, dimethylaminoethyloxy, diethylaminopropyloxy, and the like.

“Aminoalkylamino” means a —NRR′ radical where R is hydrogen or alkyl and R′ is aminoalkyl, each as defined above e.g., aminoethylamino, methylaminopropylamino, dimethylaminoethylamino, diethylaminopropylamino, and the like.

“Aminocarbonyl” means a —CONH₂ radical.

“Alkylaminocarbonyl” means a —CONHR radical where R is alkyl as defined above, e.g., methylaminocarbonyl, ethylaminocarbonyl and the like.

“Aminosulfonyl” means a —SO₂NH₂ radical.

“Aminosulfonylalkyl” means a -(alkylene)SO₂NRR′ radical where R is hydrogen or alkyl and R′ is hydrogen, alkyl, or cycloalkyl, or R and R′ together with the nitrogen atom to which they are attached form heterocyclyl, as defined above, e.g., methylaminosulfonylethyl, dimethylsulfonylethyl, and the like.

“Alkylaminosulfonyl” means a —SO₂NHR radical where R is alkyl as defined above, e.g., methylaminosulfonyl, ethylaminosulfonyl and the like.

“Aminocarbonylalkyl” means a -(alkylene)-CONRR′ radical where R′ and R″ are independently hydrogen, alkyl, haloalkyl, hydroxyalkyl, or alkoxyalkyl, each as defined herein, e.g., aminocarbonylethyl, methylaminocarbonylethyl, dimethylaminocarbonylethyl, and the like.

“Aminosulfonylalkyl” means a -(alkylene)-SO₂NRR′ radical where R′ and R″ are independently hydrogen, alkyl, haloalkyl, hydroxyalkyl, or alkoxyalkyl, each as defined herein, e.g., aminosulfonylethyl, methylaminosulfonylethyl, dimethylaminosulfonylethyl, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above e.g., benzyl, phenethyl, and the like.

“Bridged cycloalkyl” means a saturated monocyclic 5- to 7-membered hydrocarbon radical in which two non-adjacent ring atoms are linked by a (CRR′)ₙ group where n is 1 to 3 and each R is independently H or methyl (also referred to herein as the bridging group). The bridged cycloalkyl is optionally substituted with one or two substituents independently selected from alkyl, halo, alkoxy, hydroxy, or cyano. Examples of bridged cycloalkyl include but are not limited to bicyclo[2.2.1]heptane, bicyclo[2.2.2]octane, etc.

“Bridged cycloalkylalkyl” means -(alkylnene)-R radical where R is bridged cycloalkyl as defined above. Examples include, but are not limited to, bicyclo[2.2.1]heptylmethyl, and the like.

“Bridged heterocyclyl” means a saturated monocyclic ring having 5 to 7 ring carbon ring atoms in which two non-adjacent ring atoms are linked by a (CRR′)ₙ group where n is 1 to 3 and each R is independently H or methyl (also may be referred to herein as “bridging” group) and further wherein one or two ring carbon atoms, including an atom in the bridging group, is replaced by a heteroatom selected from N, O, or S(O)ₙ, where n is an integer from 0 to 2. Bridged heterocyclyl is optionally substituted with one or two substituents independently selected from alkyl, halo, alkoxy, hydroxy, or cyano. Examples include, but are not limited to, 2-azabicyclo[2.2.2]octane, quinuclidine, 7-oxabicyclo[2.2.1]heptane, and the like.

“Bridged heterocyclylalkyl” means -(alkylene)-R radical where R is bridged heterocyclyl (including specific bridged heterocyclyl rings) as defined above.

“Cycloalkyl” means a monocyclic monovalent hydrocarbon radical of three to six carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Cycloalkylalkyl” means -(alkylene)-R radical where R is cycloalkyl as defined above. Examples include, but are not limited to, cyclopropylmethyl, cyclobutylmethyl, and the like.

“Cycloalkylalkyloxy” means —O—R radical where R is cycloalkylalkyl as defined above. Examples include, but are not limited to, cyclopropylmethyloxy, cyclobutylmethyloxy, and the like.

“Cycloalkyloxyalkyl” means -(alkylene)-OR radical where R is cycloalkyl as defined above. Examples include, but are not limited to, cyclopropyloxymethyl, cyclopropyloxyethyl, cyclobutyloxyethyl, and the like.

“Cycloalkylsulfonylamino” means —NRSO₂—R′ radical where R is hydrogen or alkyl and R′ is cycloalkyl, each as defined above. Examples include, but are not limited to, cyclopropylsulfonylamino, N-cyclopropylsulfonylN(CH₃), and the like.

“Cyanoalkyl” means an alkyl radical as defined above, that is substituted with a cyano group, e.g., cyanomethyl, cyanoethyl, and the like.

“Carboxy” means —COOH radical.

“Carboxyalkyl” means an alkyl radical as defined above, that is substituted with a carboxy group e.g., carboxymethyl, carboxyethyl, and the like.

“Deuteroalkyl” means alkyl radical, as defined above, wherein one to six hydrogen atoms in alkyl chain are replaced by deuterium atoms. Examples include, but are not limited to, —CD₃, —CH₂CHD₂, and the like.

“Dialkylamino” means a —NRR′ radical where R and R′ are alkyl as defined above, e.g., dimethylamino, methylethylamino, and the like.

“Dialkylaminocarbonyl” means a —CONRR′ radical where R and R′ are alkyl as defined above, e.g., dimethylaminocarbonyl, diethylaminocarbonyl and the like.

“Dialkylaminosulfonyl” means a —SO₂NRR′ radical where R and R′ are alkyl as defined above, e.g., dimethylaminosulfonyl, diethylaminosulfonyl and the like.

“Fused cycloalkyl” means a saturated monovalent hydrocarbon radical of three to six carbon atoms that is fused to phenyl or a five- or six-membered heteroaryl ring, as defined herein, and is optionally substituted with one, two, or three substituents independently selected from alkyl, halo, alkoxy, haloalkyl, haloalkoxy, hydroxy, and cyano. Examples include, but are not limited to, tetrahydronaphthyl, 4,5,6,7-tetrahydro-1H-indolyl, 4,5,6,7-tetrahydrobenzoxazolyl, and the like.

“Fused heterocyclyl” means heterocyclyl as defined herein that is fused to cycloalkyl, phenyl or a five- or six-membered heteroaryl ring, as defined herein. Fused heterocyclyl is optionally substituted with one or two substituents independently selected from alkyl, halo, alkoxy, hydroxy, or cyano. Examples include, but are not limited to, 4,5,6,7-tetrahydro-1H-pyrrolo[2,3-b]pyridinyl, 1,2,3,4-tetrahydroquinolinyl, 3,4-dihydroquinolin-2(1H)-one, and the like.

“Fused heterocyclylalkyl” means -(alkylene)-R radical where R is fused heterocyclyloxy (including specific fused heterocyclyl rings) as defined above.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to as fluoroalkoxy.

“Haloalkoxyalkyl” means an alkyl radical that is substituted with haloalkoxy, each as defined above, e.g., trifluoromethoxyethyl, and the like.

“Heteroalkylene” means a linear saturated divalent hydrocarbon radical of two to six carbon atoms or a branched saturated divalent hydrocarbon radical of three to six carbon atoms wherein one carbon atom are replaced with —O—, —NR—, —NR′CO—, —CONR′—, SO2NR′—, or —NR′SO2-, where R and R′ are independently H or alkyl as defined herein, unless stated otherwise, e.g., —CH₂O—, —OCH₂—, —(CH₂)₂O—, —O(CH₂)₂—, —(CH₂)₂NH—, —NH(CH₂)₂—, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” means a —OR radical where R is hydroxyalkyl as defined above e.g., hydroxyethyloxy, hydroxypropyloxy, and the like.

“Hydroxyalkylamino” means a —NRR′ radical where R is hydrogen or alkyl and R′ is hydroxyalkyl, each as defined above e.g., hydroxyethylamino, hydroxypropylamino, and the like.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroaralkyl” means a -(alkylene)-R radical where R is heteroaryl (including specific rings) as defined above.

“Heteroaryloxy” means —OR where R is heteroaryl (including specific rings) as defined above.

“Heteroaralkyloxy” means a —O-(alkylene)-R radical where R is heteroaryl (including specific rings) as defined above.

“Heteroarylcarbonyl” means —COR where R is heteroaryl (including specific rings) as defined above.

“Heteroarylamino” means —NRR′ where R is hydrogen or alkyl and R′ is heteroaryl (including specific rings) as defined above.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When heterocyclyl contains at least one nitrogen atom, it may be referred to herein as heterocycloamino.

“Heterocyclylalkyl” means -(alkylene)-R radical where R is heterocyclyl (including specific heterocyclyl rings) as defined above. For example, oxetanylethyl, piperidinylethyl, and the like.

“Heterocyclyloxy” means —OR radical where R is heterocyclyl (including specific heterocyclyl rings) as defined above.

“Heterocyclylalkyloxy” means —O-(alkylene)-R radical where R is heterocyclyl (including specific heterocyclyl rings) as defined above. For example, oxetanylethyloxy, piperidinylethyloxy, and the like.

“Heterocyclylcarbonyl” means —COR where R is heterocyclyl (including specific rings) as defined above.

“Heterocyclylamino” means —NRR′ radical where R is hydrogen or alkyl and R′ is heterocyclyl (including specific heterocyclyl rings) as defined above.

“Heterocyclyloxyalkyl” means -(alkylene)-OR radical where R is heterocyclyl (including specific heterocyclyl rings) as defined above. For example, oxetanyloxyethyl, piperidinyloxyethyl, and the like.

“Heterocyclyloxyalkoxy” means —O-(alkylene)-R radical where R is heterocyclyloxy (including specific heterocyclyl rings) as defined above. For example, oxetanyloxyethyloxy, piperidinyloxyethyloxy, and the like.

“Heterocyclyloxyalkylamino” means —NR-(alkylene)-R′ radical where R is hydrogen or alkyl and R′ is heterocyclyloxy (including specific heterocyclyl rings) as defined above. For example, oxetanyloxyethylamino, piperidinyloxyethylamino, and the like.

“Oxo,” as used herein, alone or in combination, refers to ═(O).

“Optionally substituted aryl” means aryl that is optionally substituted with one, two, or three substituents independently selected from alkyl, cycloalkyl, carboxy, alkoxycarbonyl, hydroxy, hydroxyalkyl, alkoxy, alkylsulfonyl, amino, alkylamino, dialkylamino, halo, haloalkyl, haloalkoxy, and cyano.

“Optionally substituted heteroaryl” means heteroaryl as defined above that is optionally substituted with one, two, or three substituents independently selected from alkyl, alkylsulfonyl, cycloalkyl, carboxy, alkoxycarbonyl, hydroxy, alkoxy, halo, haloalkyl, haloalkoxy, amino, alkylamino, dialkylamino, and cyano.

“Optionally substituted heterocyclyl” means heterocyclyl as defined above that is optionally substituted with one, two, or three substituents independently selected from alkyl, alkylsulfonyl, cycloalkyl, carboxy, alkoxycarbonyl, hydroxy, hydroxyalkyl, alkoxy, alkoxyalkyl, aminoalkyl, halo, haloalkyl, haloalkoxy, and cyano, unless stated otherwise.

“Spirocycloalkyl” means a saturated bicyclic ring having 6 to 10 ring carbon atoms wherein the rings are connected through only one atom, the connecting atom is also called the spiroatom, most often a quaternary carbon (“spiro carbon”). The spirocycloalkyl ring is optionally substituted with one or two substituents independently selected from alkyl, halo, alkoxy, hydroxy, and cyano. Representative examples include, but are not limited to, spiro[3.3]heptane, spiro[3.4]octane, spiro[3.5]nonane, spiro[4.4]nonane (1:2:1:1), and the like.

“Spirocycloalkylalkyl” means -(alkylene)-R radical where R is spirocycloalkyl (including specific spirocycloalkyl) as defined above.

“Spiroheterocyclyl” means a saturated bicyclic ring having 6 to 10 ring atoms in which one, two, or three ring atoms are heteroatom selected from N, O, or S(O)ₙ, where n is an integer from 0 to 2, the remaining ring atoms being C and the rings are connected through only one atom, the connecting atom is also called the spiroatom, most often a quaternary carbon (“spiro carbon”). Spiroheterocyclyl is optionally substituted with one or two substituents independently selected from alkyl, halo, alkoxy, hydroxy, or cyano. Examples include, but are not limited to, Representative examples include, but are not limited to, 2,6-diazaspiro[3.3]heptane, 2,6-diazaspiro[3.4]octane, 2-azaspiro[3.4]octane, 2-azaspiro[3.5]-nonane, 2,7-diazaspiro[4.4]nonane, and the like.

“Spiroheterocyclylalkyl” means -(alkylene)-R radical where R is spiroheterocyclyl (including specific spiroheterocyclyl) as defined above.

“Sulfonylamino” means a —NRSO₂R′ radical where R is hydrogen or alkyl, and R′ is alkyl, optionally substituted aryl, optionally substituted heteroaryl, or optionally substituted heterocyclyl, each group as defined herein.

“Substituted cycloalkyl” means a saturated monocyclic monovalent hydrocarbon radical of three to six carbon atoms that is substituted with one, two or three substituents where two of the three substitutents are independently selected from alkyl, halo, alkoxy, hydroxy, haloalkyl, or haloalkoxy and the third substituent is alkyl, halo, hydroxyalkyl, haloalkyl, haloalkoxy, or cyano. Examples include, but are not limited to, 3-hydroxy-3-trifluorocyclobutyl, 2,2-dimethyl-3-hydroxycyclobutyl, and the like.

“Substituted cycloalkylalkyl” means -(alkylene)-substituted cycloalkyl, each term is defined herein. Examples include, but are not limited to, 1-hydroxymethylcycloprop-1-ylmethyl, and the like.

“Ureido” means a —NHCONRR′ radical where R and R′ are independently hydrogen or alkyl, as defined above, e.g., —NHCONHmethyl, —NHCON(CH₃)₂, and the like.

“Thioureidoalkyl” means a -(alkylene)-NHSO₂NRR′ radical where R and R′ are independently hydrogen or alkyl, as defined above, e.g., -ethylene-NHSO₂NHmethyl, -propylene-NHSO₂NH₂, and the like.

### Combination Product

Provided herein is a combination product comprising a methionine adenosyltransferase II alpha (MAT2A) inhibitor, or a pharmaceutically acceptable salt thereof, and a taxane, or a pharmaceutically acceptable salt thereof. The combination product is useful for the treatment of a variety of cancers, including solid tumors. In another aspect, the combination product is useful for the treatment of any number of MAT2A-associated diseases. In yet another aspect, the combination product is also useful for the treatment of any number of MAT2A-associated and/or microtubule-associated diseases. In another aspect, the combination product is useful for the treatment of a disease or disorder treatable by inhibiting MAT2A. In another aspect, the combination product is useful for the treating MTAP-deficient tumors.

In an embodiment, provided herein is a combination of a methionine adenosyltransferase II alpha (MAT2A) inhibitor and a taxane.

The disclosure provides MAT2A inhibitors. In an embodiment, the MAT2A inhibitor is a compound of Formula I:

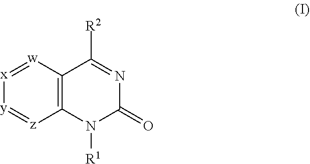

or a pharmaceutically acceptable salt thereof;
   wherein
w is CR³ or N; x is CR⁴ or N; y is CR⁵ or N; and z is CR⁶ or N, wherein:
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is hydrogen, alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ and R⁶ are independently hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that: (i) no more than two of w, x, y, and z can be N and (ii) at least one of R³, R⁴, R⁵, and R⁶ is other than hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d}, R^{e}, or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, or —X^{b}—R¹¹ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
X^{b} is a bond or alkylene; and
R¹¹ is cycloalkyl, bridged cycloalkyl, fused cycloalkyl, spirocycloalkyl, heteroaryl, heterocyclyl, bridged heterocyclyl, fused heterocyclyl, or spiroheterocyclyl, wherein heteroaryl or heterocyclyl is unsubstituted or substituted with R^{m}, Rⁿ, or R^{o}; and
R^{d}, R^{e}, R^{g}, R^{h}, R^{j}, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy.

In an embodiment, the MAT2A inhibitor is a compound of Formula I:

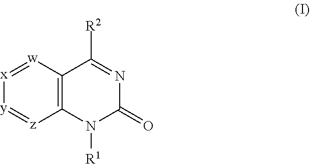

or a pharmaceutically acceptable salt thereof;
   wherein
w is CR³ or N; x is CR⁴ or N; y is CR⁵ or N; and z is CR⁶ or N, provided that no more than two of w, x, y, and z can be N, wherein:
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ and R⁶ are independently hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl;
R¹ is R⁷ wherein R⁷ is cycloalkyl, bridged cycloalkyl, fused cycloalkyl, spirocycloalkyl, aryl, heteroaryl, pyrrolidinyl, piperidinyl, piperazinyl, oxetanyl, morpholinyl, bridged heterocyclyl, fused heterocyclyl, or spiroheterocyclyl, wherein aryl, heteroaryl, or heterocyclyl is unsubstituted or substituted with R^{d}, R^{e}, or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, or —X^{b}—R¹¹ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
X^{b} is a bond or alkylene; and
R¹¹ is cycloalkyl, bridged cycloalkyl, fused cycloalkyl, spirocycloalkyl, monocyclic heteroaryl, oxetanyl, azetidinyl, 2-oxoazetidinyl, pyrrolidinyl, 2-oxopyrrolidinyl, piperidinyl, or morpholinyl, bridged heterocyclyl, fused heterocyclyl, or spiroheterocyclyl, wherein heteroaryl or heterocyclyl is unsubstituted or substituted with R^{m}, Rⁿ, or R^{o}; and
R^{d}, R^{e}, R^{g}, R^{h}, R^{j}, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, and optionally substituted heteroaryl; provided that when R¹ is pyrrolidinyl, piperidinyl, piperazinyl, oxetanyl, or morpholinyl then R^{f} is not hydroxy.

In another embodiment, the MAT2A inhibitor is a compound of Formula I:

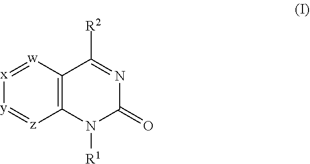

or a pharmaceutically acceptable salt thereof;
   wherein
w is CR³ or N; x is CR⁴ or N; y is CR⁵ or N; and z is CR⁶ or N, wherein:
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is hydrogen, alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ and R⁶ are independently hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that: (i) no more than two of w, x, y, and z can be N and (ii) at least one of R³, R⁴, R⁵, and R⁶ is other than hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d}, R^{e}, or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, or —X^{b}—R¹¹ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
X^{b} is a bond or alkylene; and
R¹¹ is cycloalkyl, bridged cycloalkyl, fused cycloalkyl, spirocycloalkyl, heteroaryl, heterocyclyl, bridged heterocyclyl, fused heterocyclyl, or spiroheterocyclyl, wherein heteroaryl or heterocyclyl is unsubstituted or substituted with R^{m}, Rⁿ, or R^{o}; and
R^{d}, R^{e}, R^{g}, R^{h}, R^{j}, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy;
provided that:
(1) when

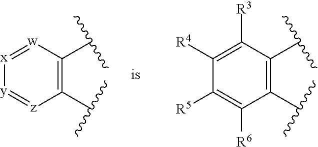

where: (a) when R² is piperazin-1-yl, 2-methylpiperazin-1-yl, or 1H-benzo[d][1,2,3]triazol-1-yl, R³ and R⁶ are hydrogen, R⁴ is chloro and R⁵ is bromo or 5-methylindazol-4-yl, then R¹ is not 2-isopropylphenyl; (b) when R² and R⁶ are methyl and R³, R⁴, and R⁵ are hydrogen; or R² and R³ are methyl and R⁴, R⁵, and R⁶ are hydrogen, then R¹ is not 2,5-, 2,6- or 2,8-dimethylquinolin-4-yl or 2-methyl-5-methoxy-, 2-methyl-6-methoxy- or 2-methyl-8-methoxyquinolin-4-yl; (c) when R² is amino or acetylamino, R⁴ is dimethylamino, and R³, R⁵, and R⁶ are hydrogen, then R¹ is not 4-hydroxy-5-hydroxymethyl-tetrahydrofuran-2-yl; (d) when R⁵ is fluoro, R³, R⁴ and R⁶ are hydrogen, and R² is 4-aminocarbonylmethyl-2-methylphenylamino, then R¹ is not 4-fluoro-2-(2-thiazol-2-ylmethoxy)phenyl, 4-fluoro-2-(2-pyridin-2-ylmethoxy)phenyl, or 4-chloro-2-methoxyphenyl; (e) when R⁶ is fluoro, R³, R⁴ and R⁵ are hydrogen, and R² is 4-aminocarbonylmethyl-2-methylphenylamino, then R¹ is not 4-fluoro-2-methoxyphenyl; (f) when R¹ is 4-chloro-2-ethoxyphenyl, R⁵ is fluoro, and R³, R⁴ and R⁶ are hydrogen, then R² is not 3-(2-oxoimidazolidin-1-yl)-2-methylphenylamino;
(2) when

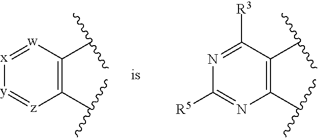

then when R¹ is 4-hydroxy-5-hydroxymethylfuran-1-yl, R¹ is amino, and R³ is methoxy; then R² is not amino; and
(3) when

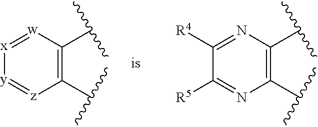

then when R¹ is 4-hydroxy-5-hydroxymethylfuran-1-yl, one of R⁴ and R⁵ is hydrogen, and the other of R⁴ and R⁵ is methyl or both of R⁴ and R⁵ are methyl, then R² is not amino.

In another embodiment,
R⁴ and R⁶ are independently hydrogen, alkyl, alkenyl, alkynyl, alkoxy, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that: (i) no more than two of w, x, y, and z can be N and (ii) at least one of R³, R⁴, R⁵, and R⁶ is other than hydrogen;
R¹⁰ is hydrogen, alkyl, deuteroalkyl, haloalkyl, hydroxyalkyl, alkoxyalkyl, aminoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l}; and
R^{f}, Rⁱ, R^{l}, and R^{o} are independently selected from alkyl, cycloalkyl, haloalkyl, haloalkoxy, alkoxy, hydroxy, halo, amino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy.

In another embodiment,
R⁴ and R⁶ are independently hydrogen, alkyl, alkenyl, alkynyl, alkoxy, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that: (i) no more than two of w, x, y, and z can be N;
R¹⁰ is hydrogen, alkyl, deuteroalkyl, haloalkyl, hydroxyalkyl, alkoxyalkyl, aminoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l}; and
R^{f}, Rⁱ, R^{l}, and R^{o} are independently selected from alkyl, cycloalkyl, haloalkyl, haloalkoxy, alkoxy, hydroxy, halo, amino, cycloalkylsulfonylamino, cyano, cyanoalkyl, alkoxycarbonylalkyl, carboxyalkyl, or —X^{c}—R¹² where X^{c} is bond, alkylene, or heteroalkylene and R¹² is optionally substituted aryl, and optionally substituted heteroaryl; provided that when R¹ is pyrrolidinyl, piperidinyl, piperazinyl, oxetanyl, or morpholinyl then R^{f} is not hydroxy.

In yet another embodiment,
R³ and R⁵ are independently hydrogen, alkyl, alkenyl, alkynyl, alkoxy, alkylsulfonyl, halo, haloalkyl, haloalkoxy, cycloalkyl, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R¹ is R⁷ wherein R⁷ is cycloalkyl, bridged cycloalkyl, fused cycloalkyl, spirocycloalkyl, phenyl, heteroaryl, heterocyclyl, bridged heterocyclyl, fused heterocyclyl, or spiroheterocyclyl, wherein phenyl, heteroaryl, or heterocyclyl is unsubstituted or substituted with R^{d}, R^{e}, or R^{f};
R⁹ is hydrogen, alkyl or cycloalkyl;
R¹⁰ is hydrogen, alkyl, haloalkyl, hydroxyalkyl, alkoxyalkyl, aminoalkyl, alkylcarbonyl, alkoxycarbonyl, alkylaminocarbonyl, dialkylaminocarbonyl, aminocarbonylalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
R^{d}, R^{e}, R^{g}, R^{h}, R^{j}, R^{k}, R^{m}, and Rⁿ are independently selected from alkyl, haloalkyl, haloalkoxy, alkoxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, haloalkyl, haloalkoxy, alkoxy, hydroxy, halo, cyano, or —X^{c}—R¹² where X^{c} is bond, alkylene or heteroalkylene and R¹² is optionally substituted aryl, optionally substituted heteroaryl, or optionally substituted heterocyclyl.

In still another embodiment, the MAT2A inhibitor is a compound of Formula (IIIa), (IIIb), (IIIc), (IIId), (IIIe), or (IIIg):

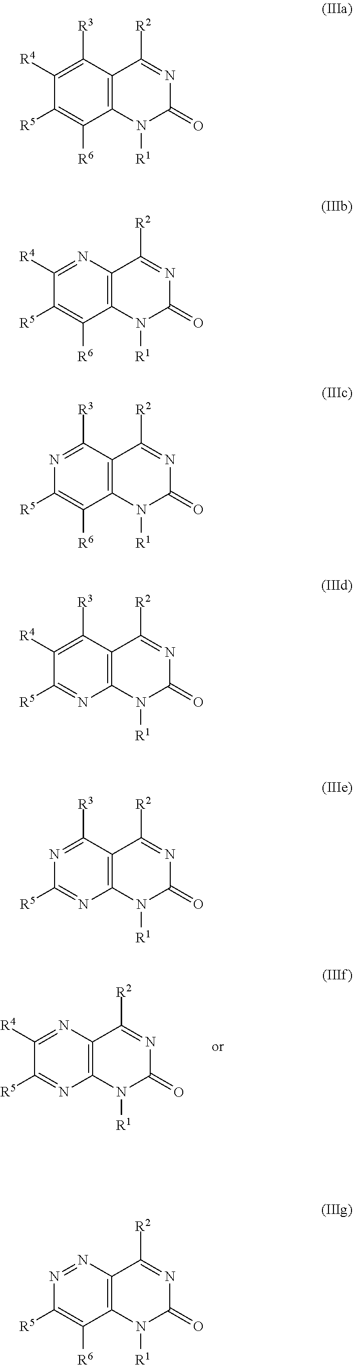

or a pharmaceutically acceptable salt thereof.

In an embodiment, the MAT2A inhibitor is a compound of Formula IIIa, or a pharmaceutically acceptable salt thereof. In another embodiment, the MAT2A inhibitor is a compound of Formula IIIb, or a pharmaceutically acceptable salt thereof. In yet another embodiment, the MAT2A inhibitor is a compound of Formula IIIc, or a pharmaceutically acceptable salt thereof. In still another embodiment, the MAT2A inhibitor is a compound of Formula IIId, or a pharmaceutically acceptable salt thereof. In an embodiment, the MAT2A inhibitor is a compound of Formula IIIe, or a pharmaceutically acceptable salt thereof. In another embodiment, the MAT2A inhibitor is a compound of Formula IIIf, or a pharmaceutically acceptable salt thereof. In yet another embodiment, the MAT2A inhibitor is a compound of Formula IIIg, or a pharmaceutically acceptable salt thereof.

In an embodiment of Formula IIId,

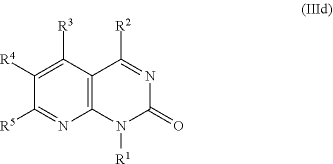

R³ is hydrogen;
R⁵ is alkyl, alkoxy, halo, haloalkyl, haloalkoxy, cycloalkyl, cyano, aminocarbonyl, heteroaryl, heterocyclyl;
R⁴ is hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{f};
R² is alkyl, haloalkyl, hydroxyalkyl, alkoxyalkyl, aminoalkyl, aminocarbonylalkyl, aminosulfonylalkyl, or —NR⁹R¹⁰, wherein:
R⁹ is hydrogen or alkyl;
R¹⁰ is hydrogen; and
R^{f} is selected from alkyl, haloalkyl, haloalkoxy, alkoxy, hydroxy, alkylsulfonyl, halo, and cyano.

In another embodiment, the MAT2A inhibitor is a compound of Formula IIIa or IIId:

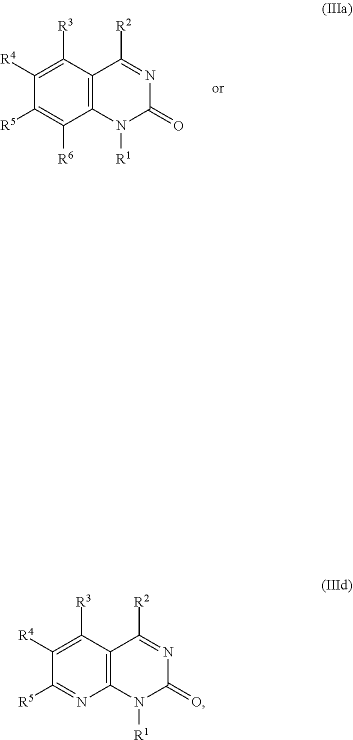

or a pharmaceutically acceptable salt thereof;
   wherein
R³ is hydrogen;
R⁵ is alkyl, alkoxy, halo, haloalkyl, haloalkoxy, cycloalkyl, cyano, aminocarbonyl, heteroaryl, heterocyclyl;
R⁴ and R⁶ are independently hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d};
R² is alkyl, haloalkyl, hydroxyalkyl, alkoxyalkyl, aminoalkyl, aminocarbonylalkyl, aminosulfonylalkyl, or —NR⁹R¹⁰, wherein:
R⁹ is hydrogen or alkyl;
R¹⁰ is hydrogen; and
R^{d} is selected from alkyl, haloalkyl, haloalkoxy, alkoxy, hydroxy, alkylsulfonyl, halo, and cyano.

In an embodiment,
R¹ is R⁷;
R⁷ is phenyl which is unsubstituted or substituted with R^{f}; and
R^{f} is selected from alkyl, haloalkyl, haloalkoxy, alkoxy, hydroxy, halo, and cyano.

In still another embodiment, R² is —NR⁹R¹⁰. In an embodiment, R² is —OR⁸. In another embodiment, R² is R¹¹.

In an embodiment, R⁵ is alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, and/or R^{c} independently selected from alkyl, cycloalkyl, haloalkyl, haloalkoxy, alkoxy, hydroxy, halo, cyano, hydroxyalkyl, alkoxyalkyl, or aminoalkyl.

In an embodiment, R¹ is R⁷ wherein R⁷ is cycloalkyl, bridged cycloalkyl, fused cycloalkyl, spirocycloalkyl, aryl, heteroaryl, pyrrolidinyl, piperidinyl, piperazinyl, oxetanyl, morpholinyl, bridged heterocyclyl, fused heterocyclyl, or spiroheterocyclyl, wherein aryl, heteroaryl, or heterocyclyl is unsubstituted or substituted with R^{d}, R^{e}, and/or R^{f}.

In an embodiment, R¹ is R⁷ wherein R⁷ is cycloalkyl, bridged cycloalkyl, fused cycloalkyl, spirocycloalkyl, aryl, heteroaryl, bridged heterocyclyl, fused heterocyclyl, or spiroheterocyclyl, wherein aryl, heteroaryl, or heterocyclyl is unsubstituted or substituted with R^{d}, R^{e}, and/or R^{f};

In yet another embodiment, R⁹ is deuteroalkyl. In still another embodiment, R⁹ is hydrogen. In an embodiment, R⁹ is alkyl. In another embodiment, R⁹ is methyl or ethyl.

In an embodiment, R¹⁰ is hydrogen, alkyl, haloalkyl, hydroxyalkyl, alkoxyalkyl, aminoalkyl, alkylcarbonyl, alkoxycarbonyl, alkylaminocarbonyl, dialkylaminocarbonyl, alkylaminocarbonylalkyl, or dialkylaminocarbonylalkyl. In another embodiment, R¹⁰ is hydrogen.

In yet another embodiment, R⁸ and R¹⁰ are alkyl. In still another embodiment, R⁸ and R¹⁰ are methyl.

In an embodiment, R⁸ and R¹⁰ are independently cycloalkyl or cycloalkylalkyl, each ring may independently be unsubstituted or substituted with one or two substituents independently selected from alkyl, halo, or cyano.

In another embodiment, R⁸ and R¹⁰ are independently cyclopropyl, cyclobutyl, 1-methylcyclopropyl, (cis)-3-hydroxy-3-methylcyclobutyl, (cis)-3-hydroxy-2,2-dimethylcyclobutyl, 1-cyanocyclobutyl, cyclopropylmethyl, 1-hydroxycyclopropmethyl, 1-fluorocyclopropmethyl, (trans)-3-hydroxy-1-methylcyclobutyl, (cis)-3-cyanocyclobutyl, 1-methylcyclobutyl, (cis)-3-hydroxycyclobutyl, (trans)-3-hydroxycyclobutyl, (trans)-3-cyanocyclobutyl, (2S,1R)-2-hydroxycyclobutyl, (1S,2S)-2-hydroxycyclobutyl, (1S,2R)-2-hydroxycyclobutyl, (1R,2R)-2-hydroxycyclobutyl, (1R,2R)-2-fluorocyclopropyl, 1-fluorocyclopropylmethyl, (1S,2R)-2-fluorocyclopropyl, (1R,2S)-2-fluorocyclopropyl, (1S,2S)-2-fluorocyclopropyl, 2,2-difluorocyclopropyl, (R)-1-cyclopropylethyl, or 2,2-difluorocyclopropylmethyl.

In yet another embodiment, R⁸ and R¹⁰ are independently cyclopropyl, cyclobutyl, 1-methylcyclopropyl, (cis)-3-hydroxy-3-methylcyclobutyl, (cis)-3-hydroxy-2,2-dimethylcyclobutyl, 1-cyanocyclobutyl, (trans)-3-hydroxy-1-methylcyclobutyl (cis)-3-cyanocyclobutyl, 1-methylcyclobutyl, (cis)-3-hydroxycyclobutyl, (trans)-3-hydroxycyclobutyl, (trans)-3-cyanocyclobutyl, (2S,1R)-2-hydroxycyclobutyl, (1S,2S)-2-hydroxycyclobutyl, (1S,2R)-2-hydroxycyclobutyl, (1R,2R)-2-hydroxycyclobutyl, (1R,2R)-2-fluorocyclopropyl, (1S,2R)-2-fluorocyclopropyl, (1R,2S)-2-fluorocyclopropyl, (1S,2S)-2-fluorocyclopropyl, or 2,2-difluorocyclopropyl.

In still another embodiment, R⁸ and R¹⁰ are independently cyclopropylmethyl, 1-hydroxycyclopropmethyl, 1-fluorocyclopropmethyl, 1-fluorocyclopropylmethyl, (R)-1-cyclopropylethyl, or 2,2-difluorocyclopropylmethyl.

In an embodiment, R⁸ and R¹⁰ are independently heteroaryl or heteroaralkyl wherein heteroaryl, by itself or as part heteroaralkyl, is unsubstituted or substituted with R^{j}, R^{k}, or R^{l}.

In another embodiment, R⁸ and R¹⁰ are heteroaryl independently selected from pyrazolyl, oxazolyl, isoxazolyl, imidazolyl, thienyl, pyrrolyl, pyridinyl, pyrimidinyl, pyrazinyl, pyridazinyl, quinolinyl, isoquinolinyl, indolyl, and indazolyl, each ring is either unsubstituted or substituted with R^{j}, R^{k}, or R^{l}.

In yet another embodiment, R⁸ and R¹⁰ are heteroaryl independently selected from pyrazolyl, imidazolyl, thienyl, pyrrolyl, pyridinyl, pyrimidinyl, pyrazinyl, pyridazinyl, quinolinyl, isoquinolinyl, indolyl, and indazolyl, each ring is either unsubstituted or substituted with R^{j}, R^{k}, or R^{l}.

In still another embodiment, R⁸ and R¹⁰ are heteroaralkyl independently selected from pyrazolylmethyl, pyrazolylethyl, oxazolylmethyl, isoxazolylmethyl, imidazolylmethyl, imidazolylethyl, thienylmethyl, thienylethyl, pyrrolylmethyl, pyrrolylethyl, pyridinylmethyl, pyridinylethyl, pyrimidinylmethyl, pyrimidinylethyl, pyrazinylmethyl, pyrazinylethyl, pyridazinylmethyl, pyridazinylethyl, quinolinylmethyl, quinolinylethyl, isoquinolinylmethyl, isoquinolinylethyl, indolylmethyl, indolylethyl, indazolylmethyl and indazolylethyl, each ring is either unsubstituted or substituted with R^{j}, R^{k}, or R^{l}.

In an embodiment, R⁸ and R¹⁰ are heteroaralkyl independently selected from pyrazolylmethyl, pyrazolylethyl, imidazolylmethyl, imidazolylethyl, thienylmethyl, thienylethyl, pyrrolylmethyl, pyrrolylethyl, pyridinylmethyl, pyridinylethyl, pyrimidinylmethyl, pyrimidinylethyl, pyrazinylmethyl, pyrazinylethyl, pyridazinylmethyl, pyridazinylethyl, quinolinylmethyl, quinolinylethyl, isoquinolinylmethyl, isoquinolinylethyl, indolylmethyl, indolylethyl, indazolylmethyl and indazolylethyl, each ring is either unsubstituted or substituted with R^{j}, R^{k}, or R^{l}.

In another embodiment, R⁸ and R¹⁰ are 1-methyl-1H-pyrazol-5-yl, isoxazol-4-yl, 3-methyl-1,2,4-oxadiazol-5-yl, 5-methylisoxazol-3-yl, 5-methylisoxazol-4-yl, 3-methoxyisoxazol-5-yl, 3,5-dimethylisoxazol-4-yl, 3-methylisoxazol-4-yl, thiazol-4-yl, thiazol-5-yl, isothiazol-4-yl, pyridin-2-yl, pyridin-3-yl, pyridin-4-yl, 2-(difluoromethyl)pyridin-4-yl, 2-(difluoromethoxy)pyridin-4-yl, 5-methoxypyridin-3-yl, 6-methylpyridin-3-yl, 6-methoxypyridin-3-yl, 3-cyanopyridin-4-yl, 3-methoxypyridin-4-yl, 3-fluoropyridin-4-yl, 3-chloropyridin-4-yl, 2-(trifluoromethyl)pyridin-4-yl, 2-methylpyridin-4-yl, pyrimidin-5-yl, 1-methyl-1H-imidazol-4-yl, 1-methylpyrazol-3-ylmethyl, 3-methoxyisoxazol-5-ylmethyl, oxazol-2-ylmethyl, oxazol-4-ylmethyl, oxazol-5-ylmethyl, isoxazol-3-ylmethyl, isoxazol-4-ylmethyl, isoxazol-5-ylmethyl, 1-methyl-1H-pyrazol-3-ylmethyl, 1-methyl-1H-pyrazol-4-ylmethyl, 1-methyl-1H-pyrazol-5-ylmethyl, pyridin-4-ylmethyl, pyridin-3-ylmethyl, or pyridin-2-ylmethyl.

In yet another embodiment,
R² is R¹¹; and
R¹¹ is heterocyclyl which is unsubstituted or substituted with R^{m}, Rⁿ, or R^{o}.

In still another embodiment, R¹¹ is oxetanyl, azetidinyl, 2-oxoazetidinyl, pyrrolidinyl, 2-oxopyrrolidinyl, piperidinyl, piperazinyl, or morpholinyl, each ring is unsubstituted or substituted with R^{m}, Rⁿ, or R^{o}.

In an embodiment, R¹¹ is cycloalkyl, bridged cycloalkyl, fused cycloalkyl, spirocycloalkyl, monocyclic heteroaryl, oxetanyl, azetidinyl, 2-oxoazetidinyl, pyrrolidinyl, 2-oxopyrrolidinyl, piperidinyl, or morpholinyl, bridged heterocyclyl, fused heterocyclyl, or spiroheterocyclyl, wherein heteroaryl or heterocyclyl is unsubstituted or substituted with R^{m}, Rⁿ, and/or R^{o}.

In an embodiment, R¹¹ is azetidin-1-yl, 4-hydroxyazetidin-1-yl, 4-methylaminocarbonylazetidin-1-yl, 4-dimethylaminocarbonylazetidin-1-yl, 2-hydromethyl-azetidin-1-yl, 2-methylazetidin-1-yl, 2-oxoazetidin-1-yl, pyrrolidin-1-yl, 2-oxopyrrolidin-1-yl, 3-hydroxypyrrolidin-1-yl, 3,3-dimethylpyrrolidin-1-yl, 3-methoxypyrrolidin-1-yl, 3-hydroxy-3-methylpyrrolidin-1-yl, piperidin-1-yl, 2-carboxypiperidin-1-yl, 2-aminocarbonylpiperidin-1-yl, piperazin-1-yl, 4-methylpiperazin-1-yl, or morpholin-4-yl.

In another embodiment,
R⁵ is alkyl, alkoxy, halo, haloalkyl, haloalkoxy, cycloalkyl, cyano, aminocarbonyl, heteroaryl, heterocyclyl, wherein heterocyclyl or heteroaryl is unsubstituted or substituted with R^{a}, R^{b}, or R^{c}; and
R^{a}, R^{b}, or R^{c} are each independently selected from alkyl, cycloalkyl, haloalkyl, haloalkoxy, alkoxy, hydroxy, halo, cyano, hydroxyalkyl, alkoxyalkyl, or aminoalkyl.

In yet another embodiment,
R¹ is methyl, ethyl, isopropyl, tert-butyl, methoxy, ethoxy, fluoro, chloro, bromo, trifluoromethyl, difluoromethyl, trifluoromethoxy, difluoromethoxy, cyclopropyl, cyclopentyl, cyano, pyrazolyl, imidazolyl, thienyl, thiazolyl, oxazolyl, isoxazolyl, pyridinyl, pyrimidinyl, oxetan-3-yl, pyrrolidin-1-yl, tetrahydrofuranyl, 2-oxoazetidin-1-yl, or 2-oxopyrrolidin-1-yl, wherein heterocyclyl or heteroaryl rings are unsubstituted or substituted with R^{a}, R^{b}, or R^{c}; and
R^{a}, R^{b}, and R^{o} are each independently selected from alkyl, cycloalkyl, haloalkyl, haloalkoxy, alkoxy, hydroxy, halo, cyano, hydroxyalkyl, alkoxyalkyl, or aminoalkyl.

In still another embodiment, R⁵ is chloro, methyl, ethyl, trifluoromethyl, 1,1-difluoroethyl, or cyclopropyl. In an embodiment, R⁵ is chloro, ethyl, or trifluoromethyl.

In another embodiment, R⁴ and R⁶ are independently selected from hydrogen, methyl, chloro, fluoro, bromo, methoxy, methylthio, methylsulfonyl, trifluoromethyl, trifluoromethoxy, cyano, amino, methylamino, dimethylamino, methylaminocarbonyl, or dimethylaminocarbonyl.

In yet another embodiment,
R⁴ is hydrogen, fluoro, bromo, methyl, methoxy, or cyano; and
R⁶ is hydrogen.

In still another embodiment, R⁴ and R⁶ are hydrogen.

In an embodiment, R³ is hydrogen, alkyl, alkoxy, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl. In another embodiment, R³ is hydrogen or methoxy. In yet another embodiment, R³ is hydrogen.

In still another embodiment, R³ is methyl, ethyl, methoxy, ethoxy, fluoro, chloro, bromo, trifluoromethyl, difluoromethyl, trifluoromethoxy, difluoromethoxy, cyclopropyl, cyano, methylsulfonyl, aminocarbonyl, methylamino, or dimethylamino.

In an embodiment,
R¹ is R⁷;
R⁷ is phenyl which is unsubstituted or substituted with R^{d}, R^{e}, or R^{f};
R^{d} and R^{e} are independently selected from alkyl, haloalkyl, haloalkoxy, alkoxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f} is selected from alkyl, haloalkyl, haloalkoxy, alkoxy, hydroxy, halo, and cyano.

In another embodiment,
R¹ is R⁷;
R⁷ is phenyl which is unsubstituted or substituted with R^{d}, R^{e}, or R^{f};
R^{d} and R^{e} are independently selected from methyl, ethyl, fluoro, chloro, bromo, methoxy, ethoxy, cyclopropyl, cyano, methylsulfonyl, methoxymethyl, aminomethyl, 2-hydroxyethyl, or 3-hydroxypropyl; and
R^{f} is selected from hydroxy, fluoro, chloro, cyano. and methyl.

In yet another embodiment,
R¹ is R⁷;
R⁷ is phenyl which is unsubstituted or substituted with R^{f}; and
R^{f} is fluoro, chloro, bromo, or methyl, wherein R^{f} is attached to carbon atoms on the phenyl ring that is ortho to the carbon atom of the phenyl ring attached to quinazolone nitrogen.

In still another embodiment,
R¹ is R⁷;
R⁷ is heteroaryl which is unsubstituted or substituted with R^{d}, R^{e}, or R^{f};
R^{d} and R^{e} are independently selected from alkyl, haloalkyl, haloalkoxy, alkoxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f} is selected from alkyl, haloalkyl, haloalkoxy, alkoxy, hydroxy, halo, and cyano.

In an embodiment,
R¹ is R⁷;
R⁷ is 5 or 6-membered heteroaryl ring which is unsubstituted or substituted with R^{d} or R^{e};
R^{d} and R^{e} are each independently selected from methyl, ethyl, fluoro, chloro, bromo, methoxy, ethoxy, cyclopropyl, cyano, methylsulfonyl, methoxymethyl, aminomethyl, 2-hydroxyethyl, and 3-hydroxypropyl.

In another embodiment, R¹ pyridinyl which is unsubstituted or substituted with R^{f}.

In an embodiment, R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, or —X^{c}—R¹² where X^{c} is bond, alkylene, or heteroalkylene and R¹² is optionally substituted aryl, and optionally substituted heteroaryl; provided that when R¹ is pyrrolidinyl, piperidinyl, piperazinyl, oxetanyl, or morpholinyl then R^{f} is not hydroxy.

In another embodiment, R^{f} is fluoro, chloro, bromo, or methyl and wherein R^{f} is attached to carbon atoms on the pyridinyl ring that is ortho to the carbon atom of the pyridinyl ring attached to quinazolone nitrogen.

In yet another embodiment, MAT2A inhibitor is selected from the group consisting of a compound from Table 7, or a pharmaceutically acceptable salt thereof.

TABLE 7

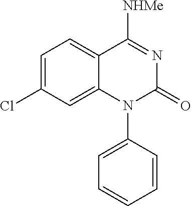 [SMILES: CNc1nc(=O)n(-c2ccccc2)c2cc(Cl)ccc12]

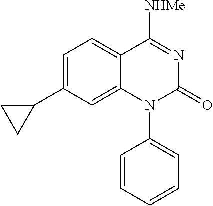 [SMILES: CNc1nc(=O)n(-c2ccccc2)c2cc(C3CC3)ccc12]

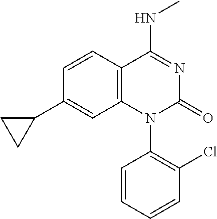 [SMILES: CNc1nc(=O)n(-c2ccccc2Cl)c2cc(C3CC3)ccc12]

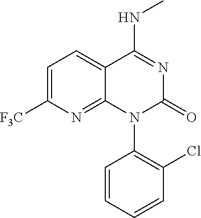 [SMILES: CNc1nc(=O)n(-c2ccccc2Cl)c2nc(C(F)(F)F)ccc12]

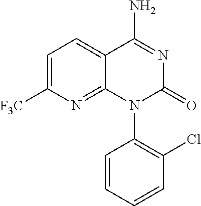 [SMILES: Nc1nc(=O)n(-c2ccccc2Cl)c2nc(C(F)(F)F)ccc12]

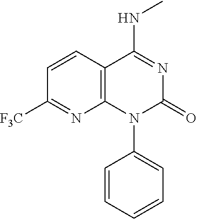 [SMILES: CNc1nc(=O)n(-c2ccccc2)c2nc(C(F)(F)F)ccc12]

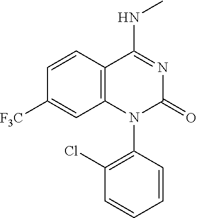 [SMILES: CNc1nc(=O)n(-c2ccccc2Cl)c2cc(C(F)(F)F)ccc12]

or a pharmaceutically acceptable salt thereof.

In another embodiment, the MAT2A inhibitor is Compound A:

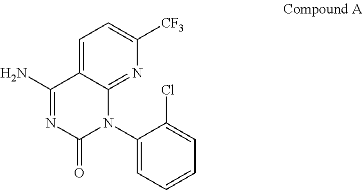 [SMILES: Nc1nc(=O)n(-c2ccccc2Cl)c2nc(C(F)(F)F)ccc12]

or a pharmaceutically acceptable salt thereof.

In another embodiment, the MAT2A inhibitor is Compound A1 having the following structural formula:

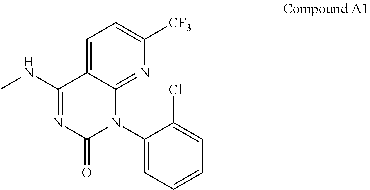 [SMILES: CNc1nc(=O)n(-c2ccccc2Cl)c2nc(C(F)(F)F)ccc12]

or a pharmaceutically acceptable salt thereof.

The preparation and activity of the MAT2A inhibitors provided herein are disclosed in PCT/US2019/065260 (WO 2020/123395), the entire contents of which are hereby incorporated by reference in their entirety.

In yet another embodiment, the taxane is selected from the group consisting of a compound in Table 8.

TABLE 8

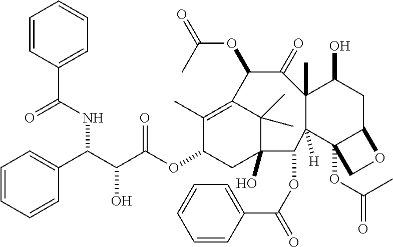

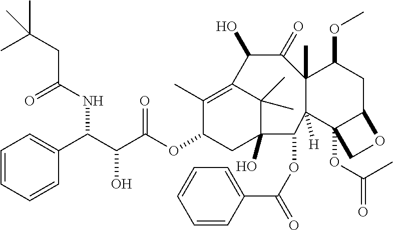

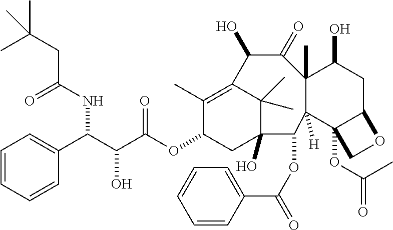

or a pharmaceutically acceptable salt or hydrate thereof.

In yet another embodiment, the taxane is protein-bound paclitaxel (Abraxane®, also referred to as Compound C).

The preparation and activity of the taxanes provided herein are described in U.S. Pat. Nos. 4,814,470; 4,857,653; 5,889,043; and 7,758,891, the entire contents of which are hereby incorporated by reference in their entirety.

In another aspect, provided herein is a combination product comprising Compound A

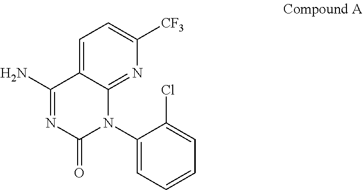 [SMILES: Nc1nc(=O)n(-c2ccccc2Cl)c2nc(C(F)(F)F)ccc12]

or a pharmaceutically acceptable salt thereof, and Compound B:

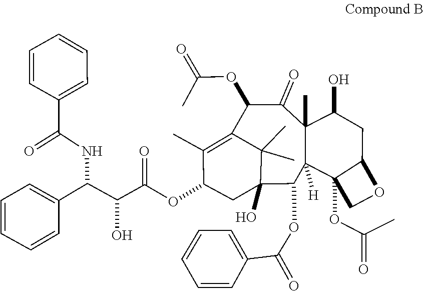 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

or a pharmaceutically acceptable salt thereof.

In another aspect, provided herein is a combination product comprising Compound

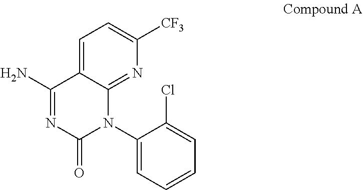 [SMILES: Nc1nc(=O)n(-c2ccccc2Cl)c2nc(C(F)(F)F)ccc12]

or a pharmaceutically acceptable salt thereof, and Compound C.

In another aspect, provided herein is a combination product comprising Compound

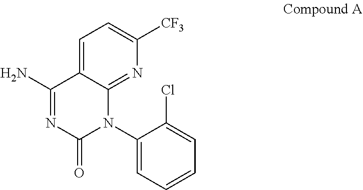 [SMILES: Nc1nc(=O)n(-c2ccccc2Cl)c2nc(C(F)(F)F)ccc12]

or a pharmaceutically acceptable salt thereof, and Compound D:

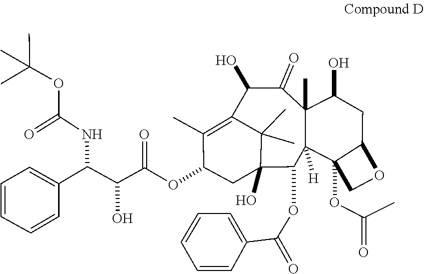 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C]

or a pharmaceutically acceptable salt thereof. In an embodiment, the Compound D is anhydrous. In an embodiment, the Compound D is a trihydrate.

In another aspect, provided herein is a combination product comprising Compound

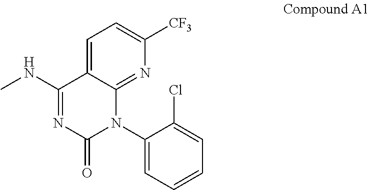 [SMILES: CNc1nc(=O)n(-c2ccccc2Cl)c2nc(C(F)(F)F)ccc12]

or a pharmaceutically acceptable salt thereof, and Compound B:

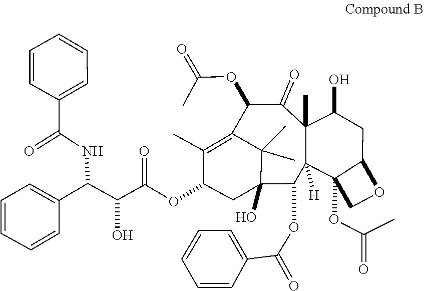 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

or a pharmaceutically acceptable salt thereof.

In another aspect, provided herein is a combination product comprising Compound A1:

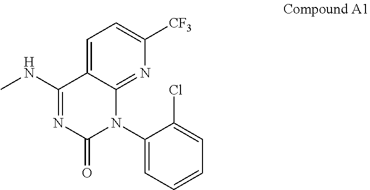 [SMILES: CNc1nc(=O)n(-c2ccccc2Cl)c2nc(C(F)(F)F)ccc12]

or a pharmaceutically acceptable salt thereof,
and Compound C.

In another aspect, provided herein is a combination product comprising Compound A1:

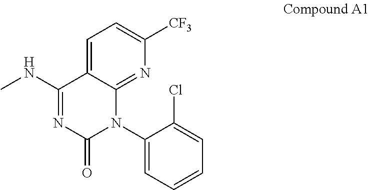 [SMILES: CNc1nc(=O)n(-c2ccccc2Cl)c2nc(C(F)(F)F)ccc12]

or a pharmaceutically acceptable salt thereof,
and Compound D:

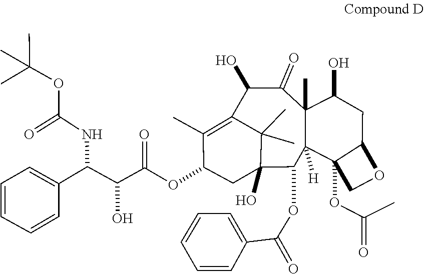 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C]

or a pharmaceutically acceptable salt thereof. In an embodiment, the Compound D is anhydrous. In an embodiment, the Compound D is a trihydrate.

The administration of a pharmaceutical combination provided herein may result in a beneficial effect, e.g. a synergistic therapeutic effect, e.g., with regard to alleviating, delaying progression of or inhibiting the symptoms, and may also result in further surprising beneficial effects, e.g., fewer side-effects, an improved quality of life or a decreased morbidity, compared with a monotherapy applying only one of the pharmaceutically active ingredients used in the combination of the invention.

### Methods of Treatment

In an embodiment, provided herein is a method of treating cancer in a subject in need thereof, the method comprising administering to the subject a therapeutically effective amount of a methionine adenosyltransferase II alpha (MAT2A) inhibitor and administering to the subject a therapeutically effective amount of a taxane, thereby treating the cancer in the subject. In an embodiment, the subject has no prior treatment with a taxane.

In an embodiment, provided herein are methods of treating cancer in a subject in need thereof, the methods comprising administering to the subject a combination comprising a methionine adenosyltransferase II alpha (MAT2A) inhibitor and a taxane, together with at least a pharmaceutically acceptable carrier, thereby treating the cancer in the subject.

In still another embodiment, the cancer is characterized by a reduction or absence of methylthioadenosine phosphorylase (MTAP) gene expression, absence of MTAP gene, reduced function of MTAP protein, reduced level or absence of MTAP protein, MTA accumulation, or combination thereof.

In an embodiment, provided herein are methods of treating cancer in a subject in need thereof, the methods comprising administering to the subject a therapeutically effective amount of a pharmaceutical composition comprising a methionine adenosyltransferase II alpha (MAT2A) inhibitor and a therapeutically effective amount of a pharmaceutical composition comprising a taxane, thereby treating the cancer in the subject.

In an embodiment, use of a combination of a MAT2A inhibitor and a taxane for the manufacture of a medicament is provided. In one embodiment, the MAT2A inhibitor is Compound A. In one embodiment, the MAT2A inhibitor is Compound A1. In one embodiment, provided is a combination of Compound A and Compound B for the manufacture of a medicament. In one embodiment, provided is a combination of Compound A and Compound C for the manufacture of a medicament. In one embodiment, provided is a combination of Compound A and Compound D for manufacture of a medicament. In one embodiment, provided is a combination of Compound A1 and Compound B for the manufacture of a medicament. In one embodiment, provided is a combination of Compound A1 and Compound C for the manufacture of a medicament. In one embodiment, provided is a combination of Compound A1 and Compound D for manufacture of a medicament. In an embodiment, the Compound D is anhydrous. In an embodiment, the Compound D is a trihydrate.

In another embodiment, use of a combination of a MAT2A inhibitor and a taxane for the treatment of cancer is provided. In one embodiment, the MAT2A inhibitor is a compound of Formula I. In one embodiment, the MAT2A inhibitor is Compound A. In one embodiment, the MAT2A inhibitor is Compound A1. In one embodiment, provided is a combination of Compound A and Compound B for the treatment of cancer. In one embodiment, provided is a combination of Compound A and Compound C for the treatment of cancer. In one embodiment, provided is a combination of Compound A and Compound D for the treatment of cancer. In one embodiment, provided is a combination of Compound A1 and Compound B for the treatment of cancer. In one embodiment, provided is a combination of Compound A1 and Compound C for the treatment of cancer. In one embodiment, provided is a combination of Compound A1 and Compound D for the treatment of cancer. In an embodiment, the Compound D is anhydrous. In an embodiment, the Compound D is a trihydrate.

In an embodiment, the MAT2A inhibitor is a compound of Formula I:

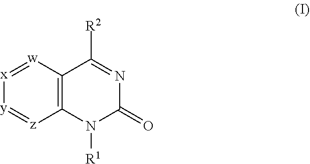

or a pharmaceutically acceptable salt thereof; wherein the variables are defined supra.

In another embodiment, the MAT2A inhibitor is a compound of Formula IIId:

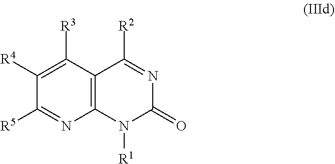

or a pharmaceutically acceptable salt thereof; wherein the variables are defined supra.

In another embodiment, the MAT2A inhibitor is a compound of Formula IIIa:

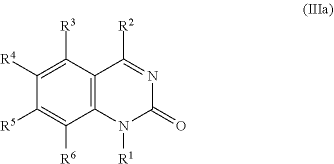

or a pharmaceutically acceptable salt thereof; wherein the variables are defined supra.

In another embodiment, the MAT2A inhibitor is selected from the group consisting of a compound from Table 7, or a pharmaceutically acceptable salt thereof.

In yet another embodiment, the MAT2A inhibitor is Compound A or a pharmaceutically acceptable salt thereof.

In yet another embodiment, the MAT2A inhibitor is Compound A1 or a pharmaceutically acceptable salt thereof.

In an embodiment, the taxane is selected from the group consisting of a compound in Table 8, or a pharmaceutically acceptable salt or hydrate thereof.

In another embodiment, the taxane is Compound B or a pharmaceutically acceptable salt thereof.

In yet another embodiment, the taxane is protein-bound paclitaxel (Abraxane®, Compound C).

In still another embodiment, the taxane is Compound D or a pharmaceutically acceptable salt thereof.

In another aspect, provided herein is a method of treating cancer in a subject in need thereof, the method comprising administering to the subject a therapeutically effective amount of Compound A or a pharmaceutically acceptable salt thereof; and administering to the subject a therapeutically effective amount of Compound B or a pharmaceutically acceptable salt thereof.

In yet another aspect, provided herein is a method of treating cancer in a subject in need thereof, the method comprising administering to the subject a therapeutically effective amount of Compound A or a pharmaceutically acceptable salt thereof; and administering to the subject a therapeutically effective amount of Compound C.

In still another aspect, provided herein is a method of treating cancer in a subject in need thereof, the method comprising administering to the subject a therapeutically effective amount of Compound A or a pharmaceutically acceptable salt thereof; and administering to the subject a therapeutically effective amount of Compound D or a pharmaceutically acceptable salt thereof. In an embodiment, the Compound D is anhydrous. In an embodiment, the Compound D is a trihydrate.

In another aspect, provided herein is a method of treating cancer in a subject in need thereof, the method comprising administering to the subject a therapeutically effective amount of Compound A1 or a pharmaceutically acceptable salt thereof; and administering to the subject a therapeutically effective amount of Compound B or a pharmaceutically acceptable salt thereof.

In yet another aspect, provided herein is a method of treating cancer in a subject in need thereof, the method comprising administering to the subject a therapeutically effective amount of Compound A1 or a pharmaceutically acceptable salt thereof; and administering to the subject a therapeutically effective amount of Compound C.

In still another aspect, provided herein is a method of treating cancer in a subject in need thereof, the method comprising administering to the subject a therapeutically effective amount of Compound A1 or a pharmaceutically acceptable salt thereof; and administering to the subject a therapeutically effective amount of Compound D or a pharmaceutically acceptable salt thereof. In an embodiment, the Compound D is anhydrous. In an embodiment, the Compound D is a trihydrate.

In another embodiment, provided is a product containing a MAT2A inhibitor and a taxane as a combined preparation for simultaneous, separate, or sequential use in medicine. In one embodiment, the MAT2A inhibitor is a compound of Formula I. In one embodiment, the MAT2A inhibitor is Compound A. In one embodiment, the MAT2A inhibitor is Compound A1. In one embodiment, provided is a product containing Compound A and Compound B as a combined preparation for simultaneous, separate, or sequential use in medicine. In one embodiment, provided is a product containing Compound A and Compound C as a combined preparation for simultaneous, separate, or sequential use in medicine. In one embodiment, provided is a product containing Compound A and Compound D as a combined preparation for simultaneous, separate, or sequential use in medicine. In one embodiment, provided is a product containing Compound A1 and Compound B as a combined preparation for simultaneous, separate, or sequential use in medicine. In one embodiment, provided is a product containing Compound A1 and Compound C as a combined preparation for simultaneous, separate, or sequential use in medicine. In one embodiment, provided is a product containing Compound A1 and Compound D as a combined preparation for simultaneous, separate, or sequential use in medicine. In an embodiment, the Compound D is anhydrous. In an embodiment, the Compound D is a trihydrate.

In another embodiment, provided is a product containing a MAT2A inhibitor and a taxane as a combined preparation for simultaneous, separate, or sequential use in treating cancer in a subject. In one embodiment, the MAT2A inhibitor is a compound of Formula I. In one embodiment, the MAT2A inhibitor is Compound A. In one embodiment, the MAT2A inhibitor is Compound A1. In one embodiment, provided is a product containing Compound A and Compound B as a combined preparation for simultaneous, separate, or sequential use in treating cancer in a subject. In one embodiment, provided is a product containing Compound A and Compound C as a combined preparation for simultaneous, separate, or sequential use in treating cancer in a subject. In one embodiment, provided is a product containing Compound A and Compound D as a combined preparation for simultaneous, separate, or sequential use in treating cancer in a subject. In one embodiment, provided is a product containing Compound A1 and Compound B as a combined preparation for simultaneous, separate, or sequential use in treating cancer in a subject. In one embodiment, provided is a product containing Compound A1 and Compound C as a combined preparation for simultaneous, separate, or sequential use in treating cancer in a subject. In one embodiment, provided is a product containing Compound A1 and Compound D as a combined preparation for simultaneous, separate, or sequential use in treating cancer in a subject. In an embodiment, the Compound D is anhydrous. In an embodiment, the Compound D is a trihydrate.

In an embodiment, provided herein is a method of inhibiting tumor growth or slowing the rate of tumor growth in a subject with an MTAP deleted cancer, comprising sequential administration of a MAT2A inhibitor and a taxane. In an embodiment, the MAT2A inhibitor is administered prior to taxane administration, or a taxane is administered prior to the MAT2A inhibitor. In another embodiment, the method comprises simultaneous administration of a MAT2A inhibitor and a taxane.

In an embodiment, tumor growth is measured by change in tumor volume from a first time point to a second time point. In some embodiments, after administering the MAT2A inhibitor and the taxane to the subject, tumor volume at the second time point shows no increase when compared to the first time point. In yet another embodiment, after administering the MAT2A inhibitor and the taxane to the subject, tumor volume decreases between the first time point and the second time point.

In an embodiment, the period of time between the first time point and the second time point can be about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; and about five years.

In another embodiment, treatment with the MAT2A inhibitor and taxane decreases the rate of tumor growth when compared to a similar time period of taxane only treatment. In an embodiment, the similar time period refers to the same or about the same amount of time of treatment.

In an embodiment, provided herein is a method of treating cancer in a subject in need thereof comprising administering a MAT2A inhibitor and a taxane to a subject who has undergone a previous cancer treatment regimen without a MAT2A inhibitor. In another embodiment, the MAT2A inhibitor and taxane are either sequentially or simultaneously administered. In yet another embodiment, the previous cancer treatment regimen comprised taxane treatment without a MAT2A inhibitor. In still another embodiment, treatment with the MAT2A inhibitor and taxane decreases the rate of tumor growth when compared to a similar time period of taxane only treatment. In another embodiment, the previous cancer treatment regimen comprised a surgical intervention.

In yet another embodiment, the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, and mesothelioma.

In an embodiment, the cancer is characterized as being MTAP-null.

In an embodiment, the cancer is characterized as being MTAP-deficient.

In still another embodiment, the cancer is a solid tumor. In still another embodiment, the cancer is a MTAP-deleted solid tumor. In still another embodiment, the cancer is a metastatic MTAP-deleted solid tumor.

In still another embodiment, the cancer is metastatic.

In still another embodiment, the cancer is MTAP-deficient lung cancer or MTAP-deficient pancreatic cancer, including MTAP-deficient NSCLC, or MTAP-deficient pancreatic ductal adenocarcinoma (PDAC), MTAP-deficient esophageal cancer, MTAP-deficient thymic cancer, MTAP-deficient adeoid cystic carcinoma, MTAP-deficient melanoma, MTAP-deficient bladder cancer, MTAP-deficient high grade sarcoma, MTAP-deficient ovarian cancer, MTAP-deficient phyllodes sarcoma, or MTAP-deficient cholangiocarcinoma. In still another embodiment, the cancer is MTAP deficient pancreatic cancer.

In still another embodiment, the cancer is a solid malignant tumor.

In another embodiment, the cancer is a tumor having an MTAP gene deletion.

In any one of the embodiments herein, the cancer is a solid tumor or a hematological cancer. In one embodiment, the tumor is deficient in MTAP. In another embodiment, the tumor is normal in its expression of MTAP.

In still another embodiment, the cancer is NSCLC, mesothelioma, squamous carcinoma of the head and neck, salivary gland tumors, urothelial cancers, sarcomas, or ovarian cancer. In still another embodiment, the cancer is NSCLC, esophagogastric and pancreatic cancers.

In still another embodiment, the cancer is characterized by a reduction or absence of MTAP gene expression, absence of MTAP gene, reduced function of MTAP protein, reduced level or absence of MTAP protein, MTA accumulation, or combination thereof.

In still another embodiment, the cancer is characterized by a reduction or absence of MTAP gene expression.

In still another embodiment, the cancer is characterized by reduced function of MTAP protein.

In still another embodiment, the cancer is characterized reduced level or absence of MTAP protein.

In still another embodiment, the cancer is characterized by MTA accumulation.

In an embodiment, the MAT2A inhibitor and the taxane are in separate dosage forms.

In another embodiment, the MAT2A inhibitor and the taxane are in the same dosage form.

In another embodiment, the treatment comprises administering the MAT2A inhibitor, or a pharmaceutically acceptable salt thereof, and the taxane, or a pharmaceutically acceptable salt thereof, at substantially the same time. In yet another embodiment, the treatment comprises administering the MAT2A inhibitor, or a pharmaceutically acceptable salt thereof, and the taxane, or a pharmaceutically acceptable salt thereof, at different times.

In still another embodiment, the MAT2A inhibitor, or a pharmaceutically acceptable salt thereof, is administered to the subject, followed by administration of the taxane, or a pharmaceutically acceptable salt thereof. In an embodiment, the taxane, or a pharmaceutically acceptable salt thereof, is administered to the subject, followed by administration of MAT2A inhibitor, or a pharmaceutically acceptable salt thereof.

In yet another embodiment, the method comprises administering to the subject in need thereof a MAT2A inhibitor.

In still another embodiment, the method comprises administering to the subject in need thereof a taxane.

In an embodiment, the MAT2A inhibitor, or a pharmaceutically acceptable salt thereof, and the taxane, or a pharmaceutically acceptable salt thereof, are administered orally.

In another embodiment, the cancer to be treated is selected from the group consisting of lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, glioma, glioblastoma, neuroblastoma, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, thymic cancer, adenoid cystic carcinoma, gastroesophageal cancer, malignant peripheral nerve sheath tumor (MPNST), and cholangiocarcinoma. In some embodiments, the cancer is lung cancer, colon cancer, breast cancer, neuroblastoma, leukemia, and lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer. In yet another embodiment, the cancer is selected from the group consisting of NSCLC, pancreatic cancer, malignant peripheral nerve sheath tumor (MPNST), and esophagogastric cancer. In yet another embodiment, the cancer is selected from the group consisting of NSCLC and pancreatic cancer. In yet another embodiment, the cancer is NSCLC. In yet another embodiment, the cancer is pancreatic cancer. In another embodiment, the cancer is phyllodes sarcoma.

In an embodiment, the cancer is a hematologic cancer, such as leukemia or lymphoma. In a certain embodiment, lymphoma is Hodgkin's lymphoma or Non-Hodgkin's lymphoma. In certain embodiments, leukemia is myeloid, lymphocytic, myelocytic, lymphoblastic, or megakaryotic leukemia.

In an aspect, provided herein is a methionine adenosyltransferase II alpha (MAT2A) inhibitor, or a pharmaceutically acceptable salt thereof, and a taxane, or a pharmaceutically acceptable salt thereof, for use in therapy.

In an embodiment, the MAT2A inhibitor, or a pharmaceutically acceptable salt thereof, and the taxane, or a pharmaceutically acceptable salt thereof, are for use in the treatment of cancer in a subject in need thereof.

Exemplary lengths of time associated with the course of the treatment methods disclosed herein include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of the MAT2A inhibitor, or a pharmaceutically acceptable salt thereof, and the amount of the taxane, or a pharmaceutically acceptable salt thereof. In an embodiment, the amount of the MAT2A inhibitor or a pharmaceutically acceptable salt thereof and the amount of taxane or a pharmaceutically acceptable salt thereof are in a single formulation or unit dosage form. In still other embodiments, the amount of MAT2A inhibitor or a pharmaceutically acceptable salt thereof and the amount of taxane or a pharmaceutically acceptable salt thereof are in a separate formulations or unit dosage forms.

In the foregoing methods, the treatment can include administering the amount of MAT2A inhibitor or a pharmaceutically acceptable salt thereof and the amount of taxane or a pharmaceutically acceptable salt thereof at substantially the same time or administering the amount of MAT2A inhibitor or a pharmaceutically acceptable salt thereof and the amount of taxane or a pharmaceutically acceptable salt thereof at different times. In some embodiments of the foregoing methods, the amount of MAT2A inhibitor or a pharmaceutically acceptable salt thereof and/or the amount of taxane or a pharmaceutically acceptable salt thereof is administered at dosages that would not be effective when one or both of MAT2A inhibitor or a pharmaceutically acceptable salt thereof and taxane or a pharmaceutically acceptable salt thereof is administered alone, but which amounts are effective in combination.

In another embodiment of the methods, the treatment includes co-administering the amount of Compound A, or a pharmaceutically acceptable salt thereof, and the amount of Compound B, or a pharmaceutically acceptable salt thereof. In an embodiment, the amount of Compound A or a pharmaceutically acceptable salt thereof and the amount of Compound B or a pharmaceutically acceptable salt thereof are in a single formulation or unit dosage form. In still other embodiments, the amount of Compound A or a pharmaceutically acceptable salt thereof and the amount of Compound B or a pharmaceutically acceptable salt thereof are in a separate formulations or unit dosage forms.

In another embodiment of the methods, the treatment includes co-administering the amount of Compound A, or a pharmaceutically acceptable salt thereof, and the amount of Compound C, or a pharmaceutically acceptable salt thereof. In an embodiment, the amount of Compound A or a pharmaceutically acceptable salt thereof and the amount of Compound C or a pharmaceutically acceptable salt thereof are in a single formulation or unit dosage form. In still other embodiments, the amount of Compound A or a pharmaceutically acceptable salt thereof and the amount of Compound C or a pharmaceutically acceptable salt thereof are in a separate formulations or unit dosage forms.

In another embodiment of the methods, the treatment includes co-administering the amount of Compound A, or a pharmaceutically acceptable salt thereof, and the amount of Compound D, or a pharmaceutically acceptable salt thereof. In an embodiment, the amount of Compound A or a pharmaceutically acceptable salt thereof and the amount of Compound D or a pharmaceutically acceptable salt thereof are in a single formulation or unit dosage form. In still other embodiments, the amount of Compound A or a pharmaceutically acceptable salt thereof and the amount of Compound D or a pharmaceutically acceptable salt thereof are in a separate formulations or unit dosage forms. In an embodiment, the Compound D is anhydrous. In an embodiment, the Compound D is a trihydrate.

In the foregoing methods, the treatment can include administering the amount of Compound A or a pharmaceutically acceptable salt thereof and the amount of Compound B or a pharmaceutically acceptable salt thereof at substantially the same time or administering the amount of Compound A or a pharmaceutically acceptable salt thereof and the amount of Compound B or a pharmaceutically acceptable salt thereof at different times. In some embodiments of the foregoing methods, the amount of Compound A or a pharmaceutically acceptable salt thereof and/or the amount of Compound B or a pharmaceutically acceptable salt thereof is administered at dosages that would not be effective when one or both of Compound A or a pharmaceutically acceptable salt thereof and Compound B or a pharmaceutically acceptable salt thereof is administered alone, but which amounts are effective in combination.

In the foregoing methods, the treatment can include administering the amount of Compound A or a pharmaceutically acceptable salt thereof and the amount of Compound C or a pharmaceutically acceptable salt thereof at substantially the same time or administering the amount of Compound A or a pharmaceutically acceptable salt thereof and the amount of Compound C or a pharmaceutically acceptable salt thereof at different times. In some embodiments of the foregoing methods, the amount of Compound A or a pharmaceutically acceptable salt thereof and/or the amount of Compound C or a pharmaceutically acceptable salt thereof is administered at dosages that would not be effective when one or both of Compound A or a pharmaceutically acceptable salt thereof and Compound C or a pharmaceutically acceptable salt thereof is administered alone, but which amounts are effective in combination.

In the foregoing methods, the treatment can include administering the amount of Compound A or a pharmaceutically acceptable salt thereof and the amount of Compound D or a pharmaceutically acceptable salt thereof at substantially the same time or administering the amount of Compound A or a pharmaceutically acceptable salt thereof and the amount of Compound D or a pharmaceutically acceptable salt thereof at different times. In some embodiments of the foregoing methods, the amount of Compound A or a pharmaceutically acceptable salt thereof and/or the amount of Compound D or a pharmaceutically acceptable salt thereof is administered at dosages that would not be effective when one or both of Compound A or a pharmaceutically acceptable salt thereof and Compound D or a pharmaceutically acceptable salt thereof is administered alone, but which amounts are effective in combination. In an embodiment, the Compound D is anhydrous. In an embodiment, the Compound D is a trihydrate.

### Non-Limiting Exemplary Embodiments

In further embodiments 1 to 39 below, the present disclosure includes:

1. In an embodiment, provided is a combination product comprising a methionine adenosyltransferase II alpha (MAT2A) inhibitor, or a pharmaceutically acceptable salt thereof, and a taxane, or a pharmaceutically acceptable salt thereof.
2. In embodiment 2, the MAT2A inhibitor of embodiment 1 is a compound of Formula I, or a pharmaceutically acceptable salt thereof.
3. In embodiment 3, the MAT2A inhibitor of embodiment 1 or 2 is selected from the group consisting of a compound in Table 7, or a pharmaceutically acceptable salt thereof.
4. In embodiment 4, the MAT2A inhibitor of embodiment 3 is Compound A or a pharmaceutically acceptable salt thereof.
5. In embodiment 5, the MAT2A inhibitor of embodiment 1 or 2 is Compound A1 or a pharmaceutically acceptable salt thereof.
6. In embodiment 6, the taxane of embodiment 1 is selected from the group consisting of a compound in Table 8, or a pharmaceutically acceptable salt thereof.
7. In embodiment 7, the taxane of embodiment 1 or 6 is Compound B or a pharmaceutically acceptable salt thereof.
8. In embodiment 8, the taxane of embodiment 1 or 6 is protein-bound paclitaxel.
9. In embodiment 8, the taxane of embodiment 1 or 6 is Compound D or a pharmaceutically acceptable salt thereof.
10. In embodiment 10, provided is a combination product comprising a first pharmaceutical composition comprising a therapeutically effective amount of a methionine adenosyltransferase II alpha (MAT2A) inhibitor and a second pharmaceutical composition comprising a therapeutically effective amount of a taxane.
11. In embodiment 11, the MAT2A inhibitor of embodiment 10 is a compound of Formula I, or a pharmaceutically acceptable salt thereof.
12. In embodiment 12, the MAT2A inhibitor of embodiment 10 or 11 is selected from the group consisting of a compound in Table 7, or a pharmaceutically acceptable salt thereof.
13. In embodiment 13, the taxane of embodiment 10 is selected from the group consisting of a compound in Table 8, or a pharmaceutically acceptable salt thereof.
14. In embodiment 14, provided is a combination product comprising a first pharmaceutical composition comprising a therapeutically effective amount of Compound A or a pharmaceutically acceptable salt thereof; and a second pharmaceutical composition comprising a therapeutically effective amount of Compound B or a pharmaceutically acceptable salt thereof.
15. In embodiment 15, provided is a combination product comprising a first pharmaceutical composition comprising a therapeutically effective amount of Compound A or a pharmaceutically acceptable salt thereof; and a second pharmaceutical composition comprising a therapeutically effective amount of protein-bound paclitaxel.
16. In embodiment 16, provided is a combination product comprising a first pharmaceutical composition comprising a therapeutically effective amount of Compound A or a pharmaceutically acceptable salt thereof; and a second pharmaceutical composition comprising a therapeutically effective amount of Compound D or a pharmaceutically acceptable salt thereof.
17. In embodiment 17, provided is a combination product comprising a first pharmaceutical composition comprising a therapeutically effective amount of Compound A1 or a pharmaceutically acceptable salt thereof; and a second pharmaceutical composition comprising a therapeutically effective amount of Compound B or a pharmaceutically acceptable salt thereof.
18. In embodiment 18, provided is a combination product comprising a first pharmaceutical composition comprising a therapeutically effective amount of Compound A1 or a pharmaceutically acceptable salt thereof; and a second pharmaceutical composition comprising a therapeutically effective amount of protein-bound paclitaxel.
19. In embodiment 19, provided is a combination product comprising a first pharmaceutical composition comprising a therapeutically effective amount of Compound A1 or a pharmaceutically acceptable salt thereof; and a second pharmaceutical composition comprising a therapeutically effective amount of Compound D or a pharmaceutically acceptable salt thereof.
20. In embodiment 20, provided is a methionine adenosyltransferase II alpha (MAT2A) inhibitor for use in treating cancer, wherein the MAT2A inhibitor is to be administered simultaneously or sequentially with a taxane.
21. In embodiment 21, the MAT2A inhibitor of embodiment 20 is a compound of Formula I, or a pharmaceutically acceptable salt thereof.
22. In embodiment 22, the MAT2A inhibitor of embodiment 20 or 21 is selected from the group consisting of a compound in Table 7, or a pharmaceutically acceptable salt thereof.
23. In embodiment 23, the taxane of embodiment 20 is selected from the group consisting of a compound in Table 8, or a pharmaceutically acceptable salt thereof.
24. In embodiment 24, provided is a methionine adenosyltransferase II alpha (MAT2A) inhibitor for use in treating cancer, wherein the MAT2A inhibitor is to be administered simultaneously or sequentially with a taxane; wherein the MAT2A inhibitor is Compound A or a pharmaceutically acceptable salt thereof; and the taxane is Compound B or a pharmaceutically acceptable salt thereof.
25. In embodiment 25, provided is a methionine adenosyltransferase II alpha (MAT2A) inhibitor for use in treating cancer, wherein the MAT2A inhibitor is to be administered simultaneously or sequentially with a taxane; wherein the MAT2A inhibitor is Compound A or a pharmaceutically acceptable salt thereof; and the taxane is protein-bound paclitaxel (Compound C).
26. In embodiment 26, provided is a methionine adenosyltransferase II alpha (MAT2A) inhibitor for use in treating cancer, wherein the MAT2A inhibitor is to be administered simultaneously or sequentially with a taxane; wherein the MAT2A inhibitor is Compound A or a pharmaceutically acceptable salt thereof; and the taxane is Compound D or a pharmaceutically acceptable salt thereof.
27. In embodiment 27, provided is a methionine adenosyltransferase II alpha (MAT2A) inhibitor for use in treating cancer, wherein the MAT2A inhibitor is to be administered simultaneously or sequentially with a taxane; wherein the MAT2A inhibitor is Compound A1 or a pharmaceutically acceptable salt thereof; and the taxane is Compound B or a pharmaceutically acceptable salt thereof.
28. In embodiment 28, provided is a methionine adenosyltransferase II alpha (MAT2A) inhibitor for use in treating cancer, wherein the MAT2A inhibitor is to be administered simultaneously or sequentially with a taxane; wherein the MAT2A inhibitor is Compound A1 or a pharmaceutically acceptable salt thereof; and the taxane is protein-bound paclitaxel (Compound C).
29. In embodiment 29, provided is a methionine adenosyltransferase II alpha (MAT2A) inhibitor for use in treating cancer, wherein the MAT2A inhibitor is to be administered simultaneously or sequentially with a taxane; wherein the MAT2A inhibitor is Compound A1 or a pharmaceutically acceptable salt thereof; and the taxane is Compound D or a pharmaceutically acceptable salt thereof.
30. In embodiment 30, provided is a use of a methionine adenosyltransferase II alpha (MAT2A) inhibitor in the manufacture of a medicament for treating cancer, wherein the MAT2A inhibitor is to be administered simultaneously or sequentially with a taxane.
31. In embodiment 31, the MAT2A inhibitor of embodiment 30 is a compound of Formula I, or a pharmaceutically acceptable salt thereof.
32. In embodiment 32, the MAT2A inhibitor of embodiment 30 or 31 is selected from the group consisting of a compound in Table 7, or a pharmaceutically acceptable salt thereof.
33. In embodiment 33, the taxane of embodiment 30 is selected from the group consisting of a compound in Table 8, or a pharmaceutically acceptable salt thereof.
34. In embodiment 34, provided is a use of a methionine adenosyltransferase II alpha (MAT2A) inhibitor in the manufacture of a medicament for treating cancer, wherein the MAT2A inhibitor is to be administered simultaneously or sequentially with a taxane; wherein the MAT2A inhibitor is Compound A or a pharmaceutically acceptable salt thereof; and the taxane is Compound B or a pharmaceutically acceptable salt thereof.
35. In embodiment 35, provided is a use of a methionine adenosyltransferase II alpha (MAT2A) inhibitor in the manufacture of a medicament for treating cancer, wherein the MAT2A inhibitor is to be administered simultaneously or sequentially with a taxane; wherein the MAT2A inhibitor is Compound A or a pharmaceutically acceptable salt thereof; and the taxane is protein-bound paclitaxel (Compound C).
36. In embodiment 36, provided is a use of a methionine adenosyltransferase II alpha (MAT2A) inhibitor in the manufacture of a medicament for treating cancer, wherein the MAT2A inhibitor is to be administered simultaneously or sequentially with a taxane; wherein the MAT2A inhibitor is Compound A or a pharmaceutically acceptable salt thereof; and the taxane is Compound D or a pharmaceutically acceptable salt thereof.
37. In embodiment 37, provided is a use of a methionine adenosyltransferase II alpha (MAT2A) inhibitor in the manufacture of a medicament for treating cancer, wherein the MAT2A inhibitor is to be administered simultaneously or sequentially with a taxane; wherein the MAT2A inhibitor is Compound A1 or a pharmaceutically acceptable salt thereof; and the taxane is Compound B or a pharmaceutically acceptable salt thereof.
38. In embodiment 38, provided is a use of a methionine adenosyltransferase II alpha (MAT2A) inhibitor in the manufacture of a medicament for treating cancer, wherein the MAT2A inhibitor is to be administered simultaneously or sequentially with a taxane; wherein the MAT2A inhibitor is Compound A1 or a pharmaceutically acceptable salt thereof; and the taxane is protein-bound paclitaxel (Compound C).
39. In embodiment 39, provided is a use of a methionine adenosyltransferase II alpha (MAT2A) inhibitor in the manufacture of a medicament for treating cancer, wherein the MAT2A inhibitor is to be administered simultaneously or sequentially with a taxane; wherein the MAT2A inhibitor is Compound A1 or a pharmaceutically acceptable salt thereof; and the taxane is Compound D or a pharmaceutically acceptable salt thereof.

### Pharmaceutical Compositions

In an aspect, provided herein is a pharmaceutical composition comprising a methionine adenosyltransferase II alpha (MAT2A) inhibitor, or a pharmaceutically acceptable salt thereof, a taxane, or a pharmaceutically acceptable salt thereof, and at least one pharmaceutically acceptable carrier.

In an embodiment, a pharmaceutical composition comprising a therapeutically effective amount of a methionine adenosyltransferase II alpha (MAT2A) inhibitor and a second pharmaceutical composition comprising a therapeutically effective amount of taxane is provided.

In another aspect, provided herein is a combination product comprising a first pharmaceutical composition comprising a therapeutically effective amount of a methionine adenosyltransferase II alpha (MAT2A) inhibitor and a second pharmaceutical composition comprising a therapeutically effective amount of a taxane.

In an embodiment, the MAT2A inhibitor is a compound of Formula I:

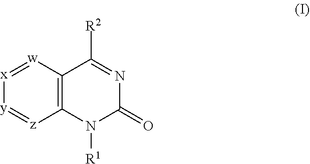

or a pharmaceutically acceptable salt thereof; wherein the variables are defined supra.

In another embodiment, the MAT2A inhibitor is selected from the group consisting of a compound from Table 7, or a pharmaceutically acceptable salt thereof.

In another embodiment, the taxane is selected from the group consisting of a compound in Table 8 or a pharmaceutically acceptable salt or hydrate thereof.

In another embodiment, the MAT2A inhibitor is Compound A:

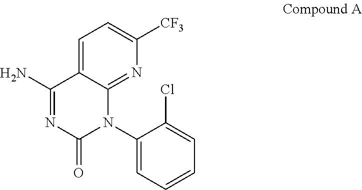 [SMILES: Nc1nc(=O)n(-c2ccccc2Cl)c2nc(C(F)(F)F)ccc12]

or a pharmaceutically acceptable salt thereof.

In another embodiment, the MAT2A inhibitor is Compound A1:

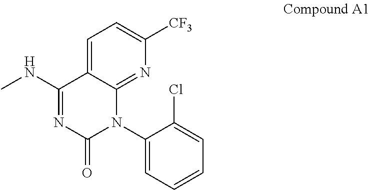 [SMILES: CNc1nc(=O)n(-c2ccccc2Cl)c2nc(C(F)(F)F)ccc12]

or a pharmaceutically acceptable salt thereof.

In another embodiment, the taxane is Compound B:

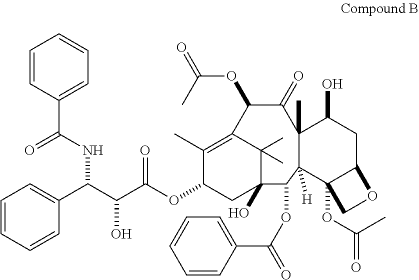

or a pharmaceutically acceptable salt thereof.

In another embodiment, the taxane is Compound C.

In another embodiment, the taxane is Compound D:

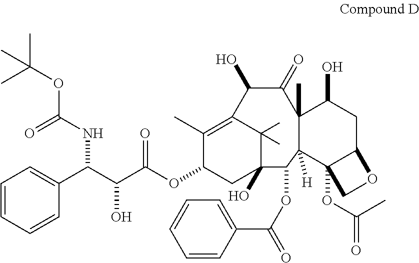

or a pharmaceutically acceptable salt thereof. In an embodiment, the Compound D is anhydrous. In an embodiment, the Compound D is a trihydrate.

In yet another aspect, provided herein is a combination product comprising a first pharmaceutical composition comprising a therapeutically effective amount of Compound A or a pharmaceutically acceptable salt thereof; and a second pharmaceutical composition comprising a therapeutically effective amount of Compound B or a pharmaceutically acceptable salt thereof.

In still another aspect, provided herein is a combination product comprising a first pharmaceutical composition comprising a therapeutically effective amount of Compound A or a pharmaceutically acceptable salt thereof; and a second pharmaceutical composition comprising a therapeutically effective amount of Compound C, or a pharmaceutically acceptable salt thereof.

In an aspect, provided herein is a combination product comprising a first pharmaceutical composition comprising a therapeutically effective amount of Compound A or a pharmaceutically acceptable salt thereof; and a second pharmaceutical composition comprising a therapeutically effective amount of Compound D or a pharmaceutically acceptable salt thereof. In an embodiment, the Compound D is anhydrous. In an embodiment, the Compound D is a trihydrate.

In yet another aspect, provided herein is a combination product comprising a first pharmaceutical composition comprising a therapeutically effective amount of Compound A1 or a pharmaceutically acceptable salt thereof; and a second pharmaceutical composition comprising a therapeutically effective amount of Compound B or a pharmaceutically acceptable salt thereof.

In still another aspect, provided herein is a combination product comprising a first pharmaceutical composition comprising a therapeutically effective amount of Compound A1 or a pharmaceutically acceptable salt thereof; and a second pharmaceutical composition comprising a therapeutically effective amount of Compound C.

In an aspect, provided herein is a combination product comprising a first pharmaceutical composition comprising a therapeutically effective amount of Compound A1 or a pharmaceutically acceptable salt thereof; and a second pharmaceutical composition comprising a therapeutically effective amount of Compound D or a pharmaceutically acceptable salt thereof. In an embodiment, the Compound D is anhydrous. In an embodiment, the Compound D is a trihydrate.

In another aspect, provided herein is a pharmaceutical composition comprising a therapeutically effective amount of Compound A or a pharmaceutically acceptable salt thereof; Compound B or a pharmaceutically acceptable salt thereof; and a pharmaceutically acceptable carrier.

In yet another aspect, provided herein is a pharmaceutical composition comprising a therapeutically effective amount of Compound A or a pharmaceutically acceptable salt thereof; Compound C, or a pharmaceutically acceptable salt thereof; and a pharmaceutically acceptable carrier.

In still another aspect, provided herein is a pharmaceutical composition comprising a therapeutically effective amount of Compound A or a pharmaceutically acceptable salt thereof; Compound D or a pharmaceutically acceptable salt thereof; and a pharmaceutically acceptable carrier. In an embodiment, the Compound D is anhydrous. In an embodiment, the Compound D is a trihydrate.

In another aspect, provided herein is a pharmaceutical composition comprising a therapeutically effective amount of Compound A1 or a pharmaceutically acceptable salt thereof; Compound B or a pharmaceutically acceptable salt thereof; and a pharmaceutically acceptable carrier.

In yet another aspect, provided herein is a pharmaceutical composition comprising a therapeutically effective amount of Compound A1 or a pharmaceutically acceptable salt thereof; Compound C; and a pharmaceutically acceptable carrier.

In still another aspect, provided herein is a pharmaceutical composition comprising a therapeutically effective amount of Compound A1 or a pharmaceutically acceptable salt thereof; Compound D or a pharmaceutically acceptable salt thereof; and a pharmaceutically acceptable carrier. In an embodiment, the Compound D is anhydrous. In an embodiment, the Compound D is a trihydrate.

In an embodiment, the pharmaceutical composition is for use in the treatment of cancer in a patient. In an embodiment, the pharmaceutical composition is for use in the treatment of a solid tumor in a patient. In an embodiment, the pharmaceutical composition is for use in the treatment of a solid malignant tumor in a patient.

### Administration/Dosage/Formulations

In another aspect, provided herein is a pharmaceutical composition or pharmaceutical combination comprising the compounds disclosed herein, together with a pharmaceutically acceptable carrier.

In an embodiment of the combination product, MAT2A inhibitor, or a pharmaceutically acceptable salt thereof, and taxane, or a pharmaceutically acceptable salt thereof, are in the same formulation. In another embodiment of the combination product, MAT2A inhibitor and taxane, are in separate formulations. In a further embodiment of this embodiment, the formulations are for simultaneous or sequential administration.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating cancer comprising a methionine adenosyltransferase II alpha (MAT2A) inhibitor, or a pharmaceutically acceptable salt thereof, and a taxane, or a pharmaceutically acceptable salt thereof.

In certain embodiments, the kit comprises a pharmaceutical product comprising a pharmaceutical composition comprising MAT2A inhibitor, or a pharmaceutically acceptable salt thereof, and a pharmaceutically acceptable carrier or diluent; and a pharmaceutical composition comprising taxane, or a pharmaceutically acceptable salt thereof, and a pharmaceutically acceptable carrier or diluent.

In some embodiments, the kit comprises a pharmaceutical composition comprising MAT2A inhibitor, or a pharmaceutically acceptable salt thereof; taxane, or a pharmaceutically acceptable salt thereof; and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g. an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment, the kit contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The kits provided herein include comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1. Efficacy of Compound a and Compound B in HCT-116 MTAP Isogenic Pair

The effect of Compound A and Compound B as single-agent anti-tumor agents and in combination was assessed in the HCT-116 human colon tumor cell line, MTAP isogenic pair (MTAP proficient (WT) or MTAP-deleted). Cells were expanded in DMEM/F12 GlutaMAX (Fisher Scientific, Catalog Number 10-565-018) with 10% fetal bovine serum. These cells were free of mycoplasma and authenticated as HCT-116 by STR profiling. Two and a half million cells in log growth phase were resuspended in Hanks Balanced Salt Solution containing 50% Matrigel and implanted subcutaneously into the flank of each recipient female CB17/Icr-Prkdc^{scid}/IcrIcoCrl mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. No dose holidays were provided for during the study and all mice were euthanized following the final dose on Day fourteen. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 9.1.0. Repeated Measures 2-Way ANOVA with Tukey's Multiple Comparisons was utilized, and P-values were presented from the final tumor measurement and were considered statistically significant if less than 0.05.

Mean tumor volume at dosing start was approximately 104 to 113 mm³, with seven mice randomized to each treatment group. The study design was identical for both models, with the study consisting of four treatment groups. Mice were dosed orally, once per day, with Vehicle, Compound A at 5 mg/kg, or dosed once weekly by intraperitoneal (IP) injection of Compound B at 15 mg/kg, or Compound A at 5 mg/kg and Compound B at 15 mg/kg. The vehicle group was a combination of Vehicle A (for Compound A, 0.5% 400 cps methylcellulose with 0.5% Tween-80 in sterile water) and Vehicle B (for Compound B, Saline).

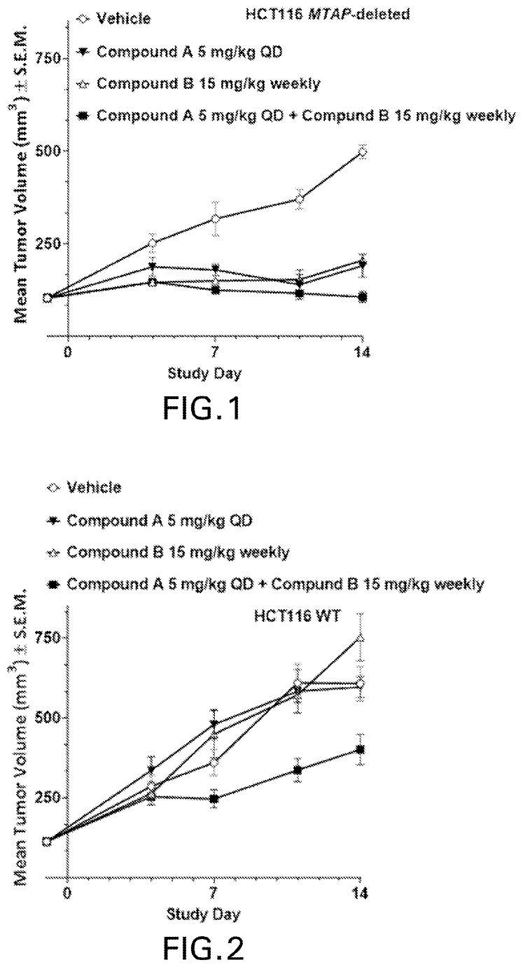

In HCT-16 MTAP-deleted, treatment with Compound A resulted in 78% TGI, while Compound B alone resulted in 74% TGI. The combination of Compound A and Compound B at resulted in 99% TGI, Table 1, FIG. 1. The combination of Compound A and Compound B prevented tumor growth in HCT-116 MTAP-deleted tumors.

TABLE 1
Summary of Efficacy of Compound A and
Compound B in HCT-116 MTAP-Deleted

| | Dose | Dose | | | | p value, | p value, |
|---|---|---|---|---|---|---|---|
| | Cmpd A | Cmpd B | Initial TV | Final TV | TGI | treated vs. | Combination |
| Model | (mg/kg) | (mg/kg) | (mean) | (mean) | (%) | Vehicle | vs. Cmpd B |
| HCT-116 | 0 | 0 | 104 | 498 | NA | NA | NA |
| MTAP- | | | | | | | |
| Deleted | | | | | | | |
| HCT-116 | 5 | 0 | 104 | 191 | 78 | 0.0004 | NA |
| MTAP- | | | | | | | |
| Deleted | | | | | | | |
| HCT-116 | 0 | 15 | 104 | 206 | 74 | <0.0001 | NA |
| MTAP- | | | | | | | |
| Deleted | | | | | | | |
| HCT-116 | 5 | 15 | 104 | 106 | 99 | <0.0001 | 0.0039 |
| MTAP- | | | | | | | |
| Deleted | | | | | | | |

In HCT-116 MTAP WT, treatment with Compound A resulted in 2% TGI, while Compound B alone resulted in −29% TGI. The combination of Compound A and Compound B resulted in 42% TGI, Table 2 FIG. 2. The combination of Compound A and Compound B did not prevent tumor growth in HCT-116 WT tumors.

TABLE 2
Summary of Efficacy of Compound A and Compound B in HCT-116 WT

| | Dose | Dose | | | | p value, | p value, |
|---|---|---|---|---|---|---|---|
| | Cmpd A | Cmpd B | Initial TV | Final TV | TGI | treated vs. | Combination |
| Model | (mg/kg) | (mg/kg) | (mean) | (mean) | (%) | Vehicle | vs. Cmpd A |
| HCT-116 | 0 | 0 | 113 | 608 | NA | NA | NA |
| MTAP | | | | | | | |
| WT | | | | | | | |
| HCT-116 | 5 | 0 | 113 | 597 | 2 | ns | NA |
| MTAP | | | | | | | |
| WT | | | | | | | |
| HCT-116 | 0 | 15 | 113 | 753 | −29 | ns | NA |
| MTAP | | | | | | | |
| WT | | | | | | | |
| HCT-116 | 5 | 15 | 113 | 402 | 42 | ns | 0.0331 |
| MTAP | | | | | | | |
| WT | | | | | | | |

ns = not statistically significant

### Example 2. Efficacy of Compound a and Compound C in Pancreatic Patient-Derived Xenograft PAXF 2094

The effect of Compound A and Compound C as single-agent anti-tumor agents and in combination was assessed in a pancreatic MTAP-deleted tumor derived from a pancreatic cancer patient and grown in recipient mice as a patient-derived xenograft model. Tumor fragments were obtained from xenografts in serial passage from nude mice. After removal from donor mice, tumors were cut into fragments (3-4 mm edge length) and placed in PBS containing 10% penicillin/streptomycin. Recipient animals were anesthetized by inhalation of isoflurane and received unilateral or bilateral tumor implants subcutaneously in the flank.

Tumor Volume (TV) was calculated using the following formula: TV (mm³)=(width×width×length)/2. No dose holidays were provided for during the study and all mice were euthanized following the final dose on Day 35. Tumor growth inhibition (TGI) was calculated by [(TV control_{final}−TV treated_{final})/(TV control_{final}−TV controlᵢₙᵢₜᵢₐₗ)×100]. TV was analyzed for statistical significance using GraphPad Prism version 9.1.0. Repeated Measures 2-Way ANOVA with Tukey's Multiple Comparisons was utilized, and P-values were presented from the final tumor measurement and were considered statistically significant if less than 0.05.

This experiment consisted of six groups with nine mice each, of which Group 1 was treated for reference with the control vehicle. Animals received monotherapies with Compound A at 3/15 mg/kg/day (Groups 2 and 3) and with Compound C at 10 mg/kg (Group 4). Combination therapies thereof were administered in Groups 5 and 6 accordingly.

Compound A was administered daily via oral gavage (p.o.), whereas animals received therapy with Compound C via intravenous injection (i.v.) once per week over the course of five weeks (35 days). The treatment started when the mean tumor volume reached about 131-134 mm³. Mice were then randomly assigned to respective groups so that the mean starting tumor volume was the same among groups.

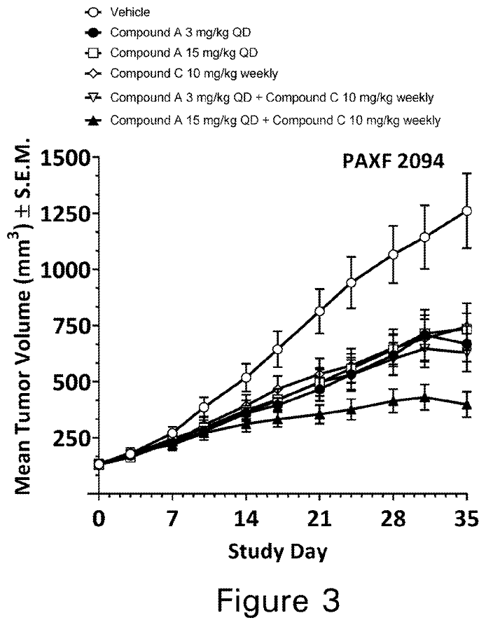

Treatment with Compound A resulted in 47-52% TGI, 46% TGI with Compound C, 56% TGI with Compound A at 3 mg/kg and Compound C, and 76% TGI with Compound A at 15 mg/kg and Compound C, Table 3, FIG. 3. The combination of Compound A and Compound C resulted in a significant delay in tumor growth when compared to Compound C alone.

TABLE 3
Summary of Efficacy of Compound A and Compound C in PAXF 2094

| | Dose | Dose | | | | p value, | p value, |
|---|---|---|---|---|---|---|---|
| | Cmpd A | Cmpd C | Initial TV | Final TV | TGI | treated vs. | Combination |
| Model | (mg/kg) | (mg/kg) | (mean) | (mean) | (%) | Vehicle | vs. Cmpd C |
| PAXF | 0 | 0 | 131 | 1261 | NA | NA | NA |
| 2094 | | | | | | | |
| PAXF | 3 | 0 | 131 | 668 | 52 | <0.0001 | NA |
| 2094 | | | | | | | |
| PAXF | 15 | 0 | 131 | 735 | 47 | <0.0001 | NA |
| 2094 | | | | | | | |
| PAXF | 0 | 10 | 131 | 745 | 46 | <0.0001 | NA |
| 2094 | | | | | | | |
| PAXF | 3 | 10 | 134 | 629 | 56 | <0.0001 | ns |
| 2094 | | | | | | | |
| PAXF | 15 | 10 | 133 | 398 | 76 | <0.0001 | 0.0017 |
| 2094 | | | | | | | |

ns = not statistically significant

### Example 3. Efficacy of Compound a and Compound B in NCI-H838 NSCLC Xenograft

The effect of Compound A and Compound B as single-agent anti-tumor agents and in combination was assessed in the NCI-H838 human NSCLC tumor cell line, an endogenously MTAP-deleted cell line. Cells were expanded in RPMI with 10% fetal bovine serum. These cells were free of mycoplasma and authenticated by STR profiling. Five million cells in log growth phase were resuspended in Hanks Balanced Salt Solution containing 50% Matrigel and implanted subcutaneously into the flank of each recipient female NOD SCID mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. No dose holidays were provided for during the study and all mice were euthanized following the final dose on Day twentyfour. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 9.1.0. Repeated Measures 2-Way ANOVA with Tukey's Multiple Comparisons was utilized, and P-values were presented from the final tumor measurement and were considered statistically significant if less than 0.05.

Mean tumor volume at dosing start was approximately 120 mm³, with eight mice randomized to each treatment group, with the study consisting of six treatment groups. Mice were dosed orally, once per day, with Vehicle, Compound A at 3 mg/kg or 30 mg/kg or dosed once weekly by intraperitoneal (IP) injection of Compound B at 15 mg/kg; or Compound A at 3 mg/kg or 30 mg/kg and Compound B at 15 mg/kg. The vehicle group was a combination of Vehicle A (for Compound A, 0.5% 400 cps methylcellulose with 0.5% Tween-80 in sterile water) and Vehicle B (for Compound B, Saline).

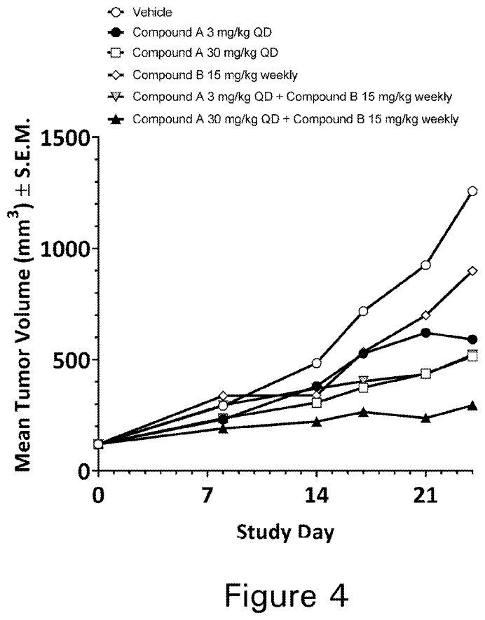

Treatment with Compound A resulted in 59-65% TGI, while Compound B alone resulted in 32% TGI. The combination of Compound A and Compound B resulted in 65-85% TGI, Table 4, FIG. 4. The combination of Compound A and Compound B delayed tumor growth.

TABLE 4
Efficacy of Compound A and Compound B in NCI-H838

| | Dose | Dose | | | | p value, | p value, |
|---|---|---|---|---|---|---|---|
| | Cmpd A | Cmpd B | Initial TV | Final TV | TGI | treated vs. | Combination |
| Model | (mg/kg) | (mg/kg) | (mean) | (mean) | (%) | Vehicle | vs. Cmpd B |
| NCI- | 0 | 0 | 120 | 1258 | NA | NA | NA |
| H838 | | | | | | | |
| NCI- | 3 | 0 | 120 | 591 | 59 | <0.0001 | NA |
| H838 | | | | | | | |
| NCI- | 30 | 0 | 120 | 515 | 65 | <0.0001 | NA |
| H838 | | | | | | | |
| NCI- | 0 | 15 | 120 | 899 | 32 | 0.0001 | NA |
| H838 | | | | | | | |
| NCI- | 3 | 15 | 120 | 522 | 65 | <0.0001 | <0.0001 |
| H838 | | | | | | | |
| NCI- | 30 | 15 | 120 | 294 | 85 | <0.0001 | <0.0001 |
| H838 | | | | | | | |

### Example 4. Efficacy of Compound a and Compound B or Compound D in NCI-H838 NSCLC Xenograft

The effect of Compound A and Compound B or Compound D as single-agent anti-tumor agents and in combination was assessed in the NCI-H838 human NSCLC tumor cell line.

Mean tumor volume at dosing start was approximately 120 mm³, with eight mice randomized to each treatment group, with the study consisting of six treatment groups. Mice were dosed orally, once per day, with Vehicle, Compound A at 30 mg/kg, or dosed once weekly by intraperitoneal (IP) injection of Compound B at 15 mg/kg or Compound D at 10 mg/kg, or combination of Compound A and Compound B or Compound A and Compound D. The vehicle group was a combination of Vehicle A (for Compound A, 0.5% 400 cps methylcellulose with 0.5% Tween-80 in sterile water) and Vehicle B (for Compound B and D, Saline).

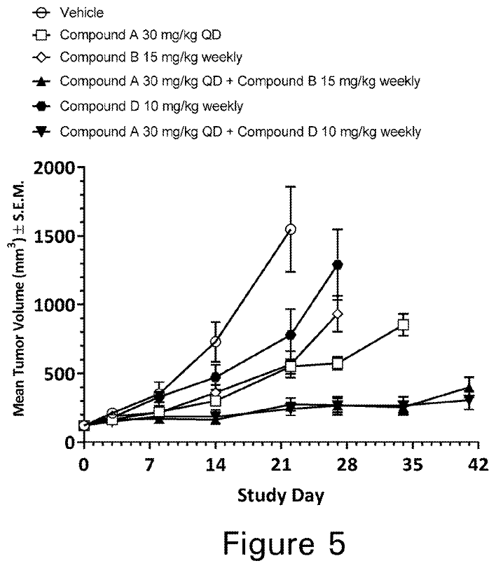

On Study Day 22, treatment with Compound A resulted in 70% TGI, while Compound B alone resulted in 69% TGI and Compound D alone resulting in 54% TGI. The combination of Compound A and Compound B resulted in 89% TGI, and Compound A and Compound D resulted in 92% TGI, Table 5 and FIG. 5. Both combinations resulted in tumor stabilization and significant TGI.

TABLE 5
Efficacy of Compound A and Compound B or Compound D in NCI-H838

| | | | | | Final | | p value, | p value, |
|---|---|---|---|---|---|---|---|---|
| | Dose | Dose | Dose | Initial | TV on | TGI on | treated vs. | Combination |
| | Cmpd A | Cmpd B | Cmpd D | TV | Day 22 | Day 22, | Vehicle | vs. Cmpd |
| Model | (mg/kg) | (mg/kg) | (mg/kg) | (mean) | (mean) | (%) | (Day 22) | (Day 27) |
| NCI- | 0 | 0 | 0 | 120 | 1547 | NA | NA | NA |
| H838 | | | | | | | | |
| NCI- | 30 | 0 | 0 | 120 | 549 | 70 | <0.0001 | NA |
| H838 | | | | | | | | |
| NCI- | 0 | 15 | 0 | 120 | 566 | 69 | <0.0001 | NA |
| H838 | | | | | | | | |
| NCI- | 30 | 15 | 0 | 120 | 274 | 89 | 0.0001 | 0.0048 |
| H838 | | | | | | | | |
| NCI- | 0 | 0 | 10 | 120 | 780 | 54 | <0.0001 | ns |
| H838 | | | | | | | | |
| NCI- | 30 | 0 | 10 | 120 | 241 | 92 | <0.0001 | 0.0206 |
| H838 | | | | | | | | |

### Example 5. Efficacy of Compound a and Compound B or Compound D in NCI-H647 NSCLC Xenograft

The effect of Compound A, Compound B, or Compound D as single-agent anti-tumor agents and in combination was assessed in the NCI-H647 human NSCLC tumor cell line, an endogenously MTAP-deleted cell line. Cells were expanded in RPMI with 10% fetal bovine serum. These cells were free of mycoplasma and authenticated by STR profiling. Four million cells in log growth phase were resuspended in Hanks Balanced Salt Solution containing 50% Matrigel and implanted subcutaneously into the flank of each recipient female NOD SCID mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. No dose holidays were provided for during the study and all mice were euthanized following the final dose on Day thirty-five. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 9.1.0. Repeated Measures 2-Way ANOVA with Tukey's Multiple Comparisons was utilized, and P-values were presented from the final tumor measurement and were considered statistically significant if less than 0.05. Mean tumor volume at dosing start was approximately 143-144 mm³, with eight mice randomized to each treatment group, with the study consisting of nine treatment groups. Mice were dosed orally, once per day, with Vehicle, Compound A at 3 mg/kg or 30 mg/kg, or dosed once weekly by intraperitoneal (IP) injection of Compound B at 15 mg/kg or Compound D at 10 mg/kg, or Compound A at 3 mg/kg or 30 mg/kg and Compound B or Compound D. The vehicle group was a combination of Vehicle A (for Compound A, 0.5% 400 cps methylcellulose with 0.5% Tween-80 in sterile water) and Vehicle B (for Compound B and D, Saline).

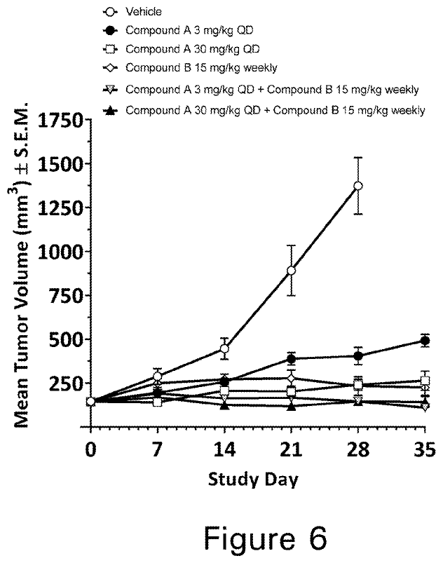

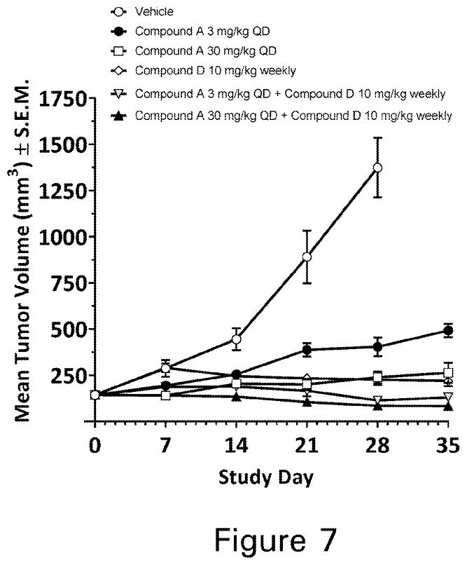

Treatment with Compound A resulted in 79-92% TGI, while Compound B or Compound D alone resulted in 93% TGI, Table 6. The combination of Compound A and Compound B resulted in 100% TGI, FIG. 6, and Compound A and Compound D resulted in 102-105% TGI, FIG. 7. The combination of Compound A with Compound B or Compound D resulted in tumor stasis or tumor regression.

TABLE 6
Efficacy of Compound A and Compound B or Compound D in NCI-H647

| | | | | | | | p value, | |
|---|---|---|---|---|---|---|---|---|
| | | | | | Final TV | | treated | p value, |
| | Dose | Dose | Dose | Initial | on Day | | vs. | Combination |
| | Cmpd A | Cmpd B | Cmpd D | TV | 28 | TGI | Vehicle | vs. Cmpd B |
| Model | (mg/kg) | (mg/kg) | (mg/kg) | (mean) | (mean) | (%) | (Day 28) | or D (Day 35) |
| NCI- | 0 | 0 | 0 | 144 | 1373 | NA | NA | NA |
| H647 | | | | | | | | |
| NCI- | 3 | 0 | 0 | 144 | 404 | 79 | <0.0001 | NA |
| H647 | | | | | | | | |
| NCI- | 30 | 0 | 0 | 144 | 240 | 92 | <0.0001 | NA |
| H647 | | | | | | | | |
| NCI- | 0 | 15 | 0 | 144 | 233 | 93 | <0.0001 | NA |
| H647 | | | | | | | | |
| NCI- | 3 | 15 | 0 | 144 | 145 | 100 | <0.0001 | 0.0049 |
| H647 | | | | | | | | |
| NCI- | 30 | 15 | 0 | 143 | 145 | 100 | <0.0001 | ns |
| H647 | | | | | | | | |
| NCI- | 0 | 0 | 10 | 144 | 226 | 93 | <0.0001 | NA |
| H647 | | | | | | | | |
| NCI- | 3 | 0 | 10 | 143 | 114 | 102 | <0.0001 | 0.0441 |
| H647 | | | | | | | | |
| NCI- | 30 | 0 | 10 | 143 | 85 | 105 | <0.0001 | 0.0004 |
| H647 | | | | | | | | |

Compound A was found to provide statistically significant anti-tumor activity in four MTAP-deleted xenograft models. Compound A when combined with Compound B prevented tumor growth in the HCT-116 MTAP-deleted model but not the HCT-116 MTAP WT model. In the Pancreatic PDX model, PAXF 2094, 15 mg/kg Compound A with Compound D resulted in significant benefit over Compound D alone. In the NSCLC models NCI-H838 and NCI-H647, Compound A combination with Compound B or Compound D provided greater anti-tumor activity than Compound B or Compound D monotherapy. In NCI-H647, tumor stasis and tumor regressions were achieved with the combination of Compound A with Compound B or Compound D, respectively.

Accordingly, when Compound A is combined with taxane chemotherapies, the combination provides significant anti-tumor activity.

### Example 6. MAT2A Inhibition and Taxanes Demonstrate Synergistic Growth Inhibition in In Vitro MTAP-Deficient Models

### Materials and Methods

A panel of 13 MTAP-deficient non-small cell lung cancer (NSCLC), bladder, pancreatic, gastric, esophageal, and head and neck squamous cell carcinoma (HNSCC) cancer cell lines was used to assess the combinatory effect of a MAT2A inhibitor (Compound A) and taxanes (Paclitaxel (Compound B) and Docetaxel (Compound D)) using Horizon Discovery's High Throughput Screening platform. To determine the optimal concentration range of each compound in each cell line for the combination screen, cells were seeded at 150 cells/well for Compound A or 500 cells/well for Taxanes in a 384-well plate, and 24 hrs later, treated with a 9-point titration of each compound as a single agent for 6 days. For the combination screen, cells were seeded at 150 cells/well in a 384-well plate, and 24 hrs later, co-treated with a 5-point titration of each compound in an optimized 6×6 dose matrix for 6 days. Cells were subsequently lysed with Promega CellTiter-Glo (CTG) 2.0 reagent according to the manufacturer's protocol and the chemiluminescent signal was measured using a PerkinElmer EnVision plate reader for cell number quantification. A plate of untreated cells was harvested at the time of compound addition (T₀ or time zero) for cell number quantification using CTG. Each data point was run in technical triplicate.

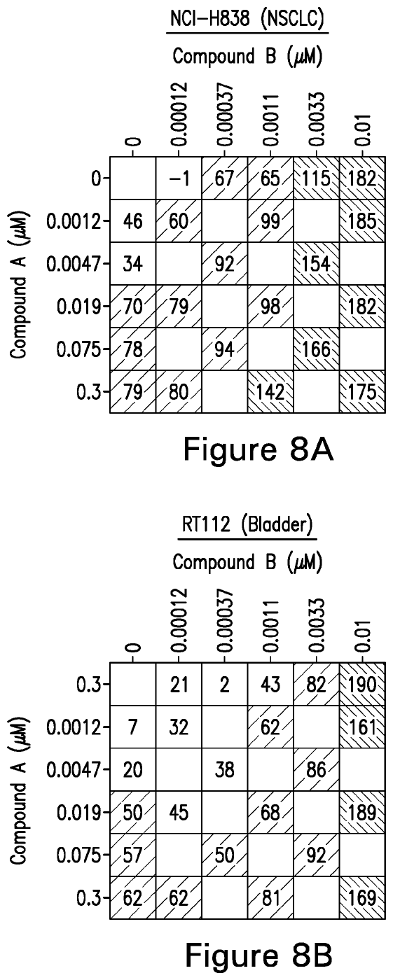

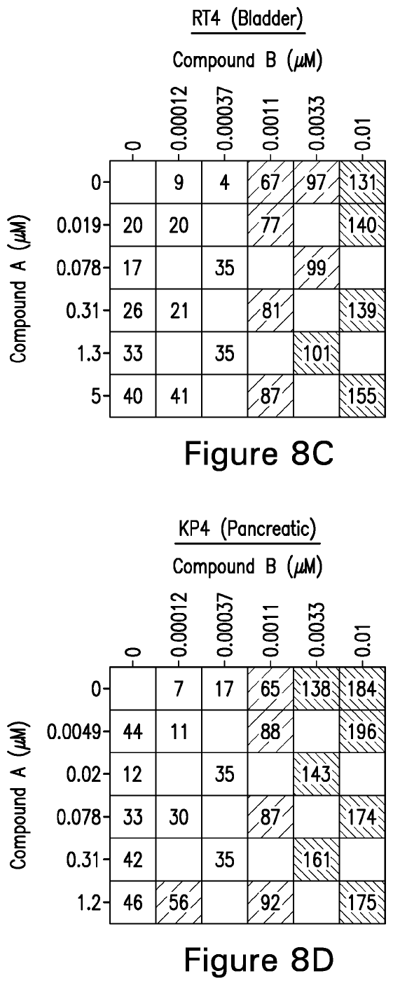

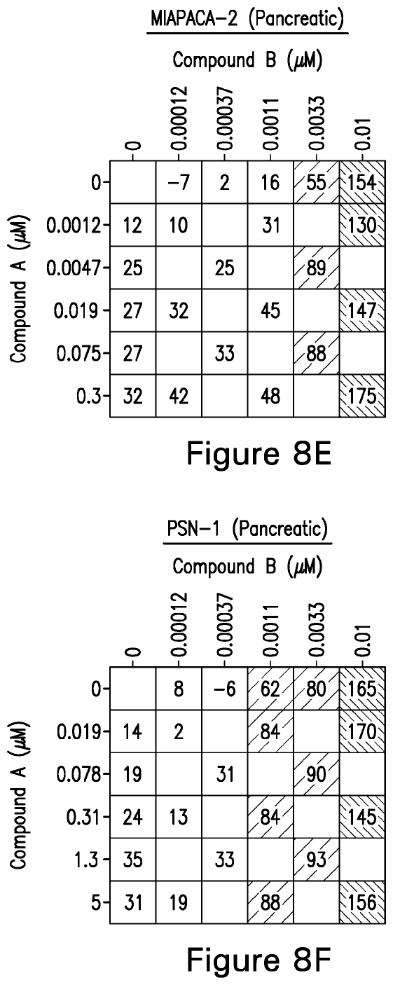

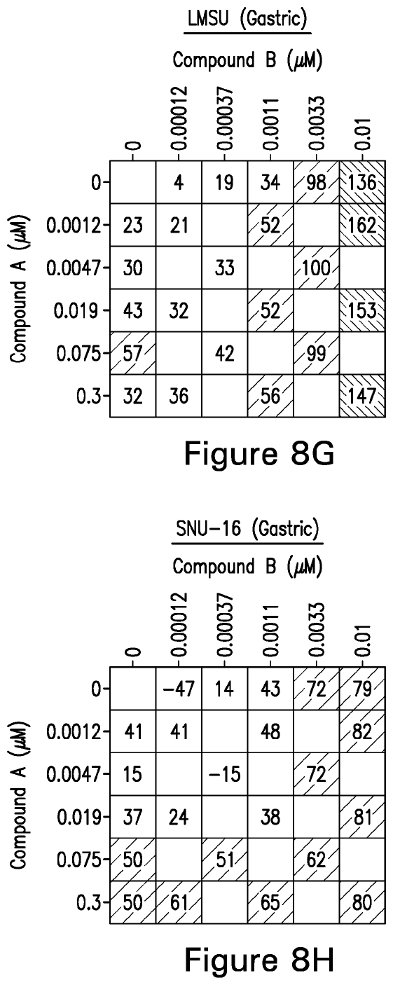

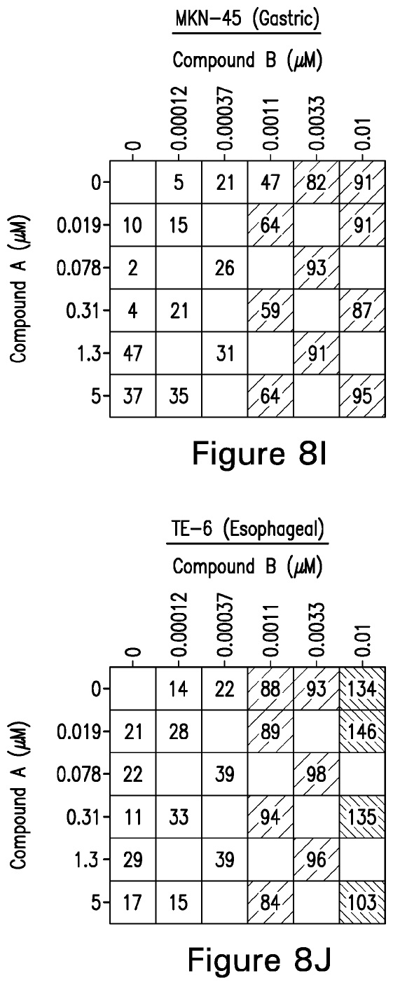

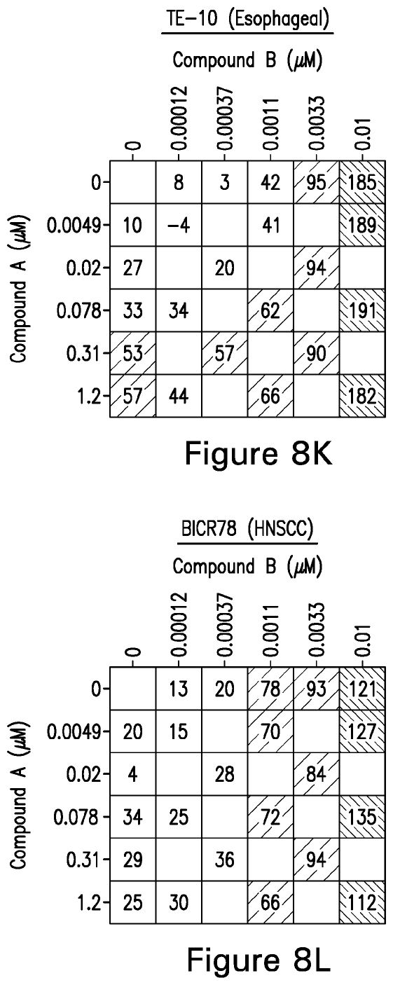

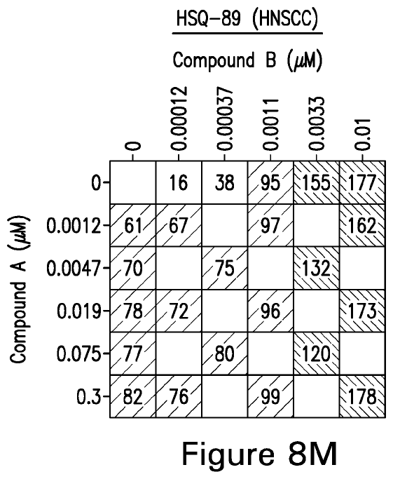

The percent of growth inhibition is calculated as:

$$If T < {V}_{0} : 100 * ( 1 - ( T - {V}_{0} ) / {V}_{0} )$$

$$If T \geq {V}_{0} : 100 * ( 1 - ( T - {V}_{0} ) / ( V - {V}_{0} ) )$$

where T is the signal measure for a test article, V is the vehicle-treated control measure, and V₀ is the vehicle control measure at T₀). This formula is derived from the growth inhibition calculation used in the National Cancer Institute's NCI-60 high throughput screen. The percent growth inhibition is used to generate dose-response curves and GI₅₀ calculations for single drug activity and drug combination synergy in Horizon's Chalice Analyzer software. 100% and >100% growth inhibition represented cytostasis and cytotoxicity, respectively. Growth inhibition is presented as a percent of TO for each cell line in a 6×6 dose matrix. 50-100% growth inhibition is highlighted in light gray, with 100% growth inhibition representing cytostasis in FIGS. 8A-M and 10A-M. Greater than 100% growth inhibition is highlighted in dark gray and represents cytotoxicity.

Synergistic growth inhibition was assessed using the Loewe additivity model. The observed growth inhibition at each dose was compared to the predicted inhibition based on the additive activity of either drug combined with itself. The calculation for Loewe additivity is: I_{Loewe} that satisfies (X/X_{I})+(Y/Y_{I})=1, where X_{I} and Y_{I} are the single agent effective concentrations for the observed combination effect I. A difference between the observed and predicted growth inhibition 20% was considered synergistic.

The strength of synergy was quantified using Horizon's Synergy Score, which is calculated as:

$$Synergy Score = log {f}_{X} log {f}_{Y} \sum max ( 0 , {I}_{data} ) ( {I}_{data} - {I}_{Loewe} )$$

The fractional inhibition for each component agent and combination point in the matrix is calculated relative to the median of all untreated/vehicle-treated control wells. The Synergy Score equation integrates the observed growth inhibition each at point in the dose matrix in excess over predicted growth inhibition and dilution factors of individual compounds. The inclusion of positive inhibition gating or an I_{data} multiplier removes noise near the zero-effect level, and biases results for synergistic interactions at that occur at high activity levels. Combinations with higher maximum growth inhibition effects or those which are synergistic at low concentrations will have higher Synergy Scores. A Synergy Score of >3.0 was considered synergistic, given that it corresponded with high growth inhibition and 20% excess over Loewe additivity model at multiple dose points.

### Results

The combination of Compounds A and B was synergistic (Synergy Score >3) in 6/13 MTAP-deficient cell lines, NCI-H838 (NSCLC), RT4 (bladder), KP4 (pancreatic), MIAPACA-2 (pancreatic), PSN-1 (pancreatic), and LMSU (gastric) (Table 9). This is supported by the enhanced growth inhibition and cytotoxicity at multiple evaluated doses compared to the effect of either agent alone, which is reflected in the Loewe additivity model of synergy (FIGS. 1 and 2). Additionally, while this combination was synergistic across models of multiple tumor types, it was synergistic in all evaluated models of pancreatic cancer, suggesting that patients with pancreatic cancer may be sensitive to this combination therapy.

Similarly, the combination of Compound A and Compound D was synergistic in 7/13 MTAP-deficient cell lines, NCI-H838 (NSCLC), RT112 (Bladder), RT4 (Bladder), MIAPACA-2 (pancreatic), PSN-1 (pancreatic), TE-10 (Esophageal), and HSQ-89 (HNSCC) (FIG. 2 and Table 9). This corresponded to enhanced growth inhibition compared to that elicited by either single agent.

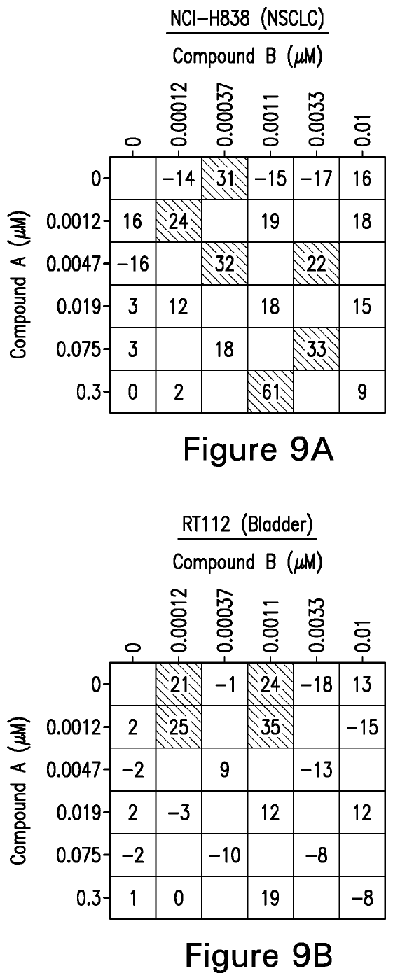

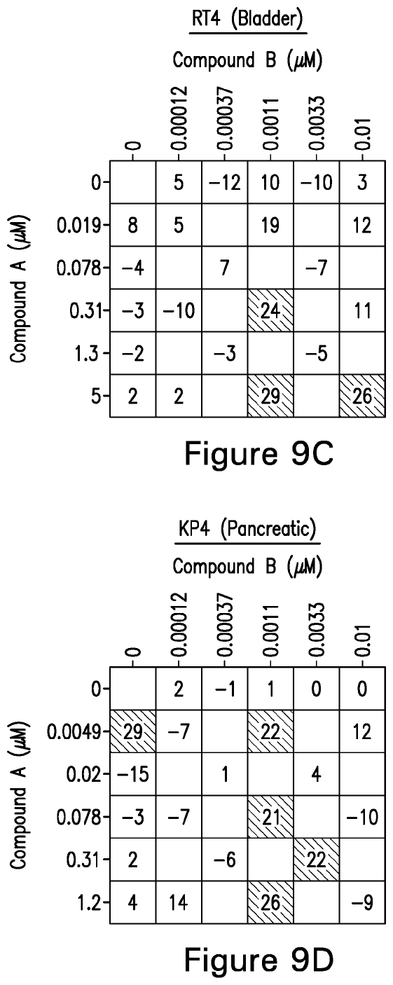

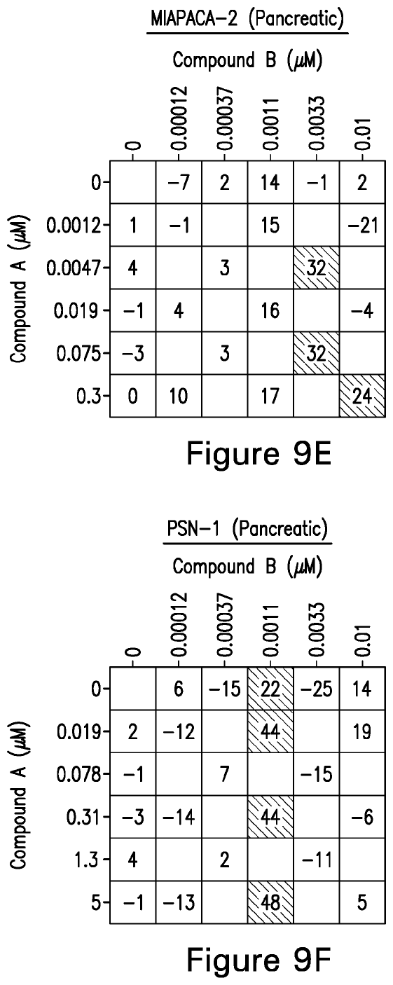

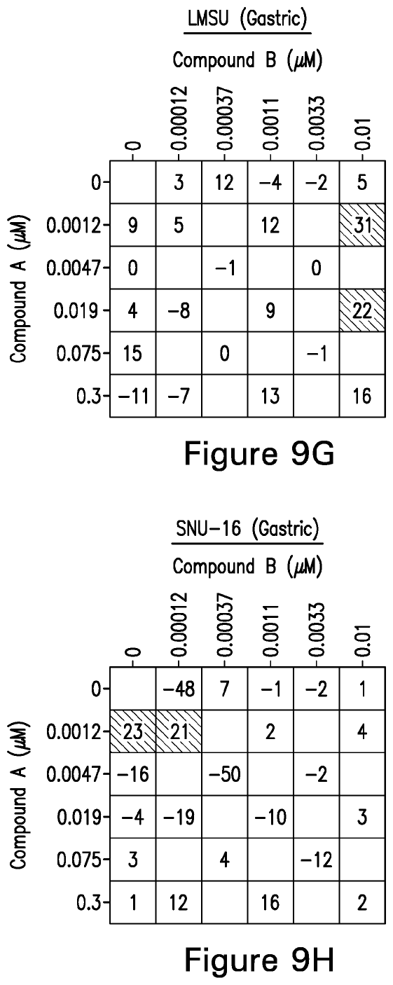

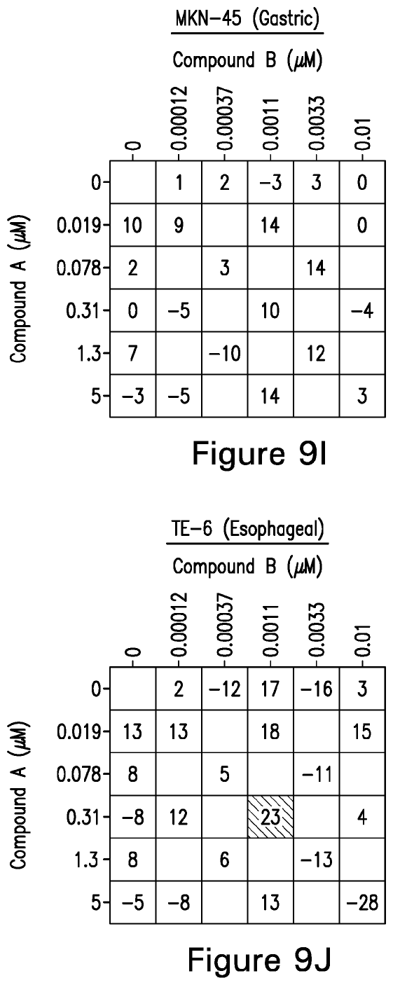

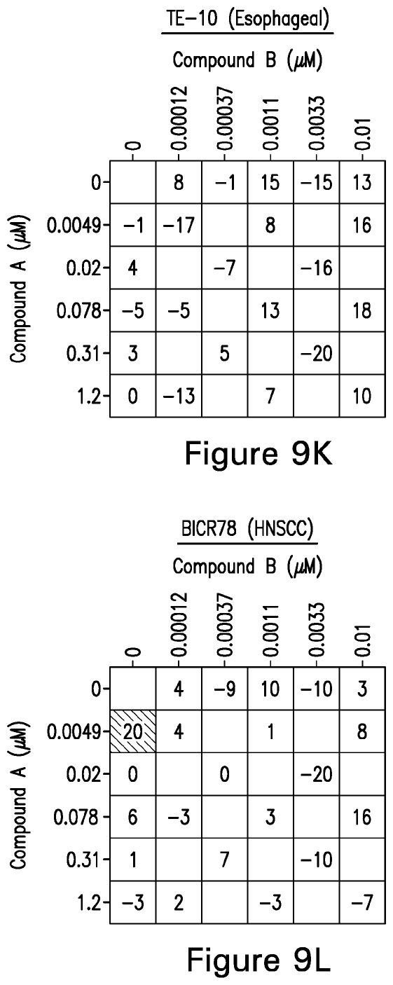

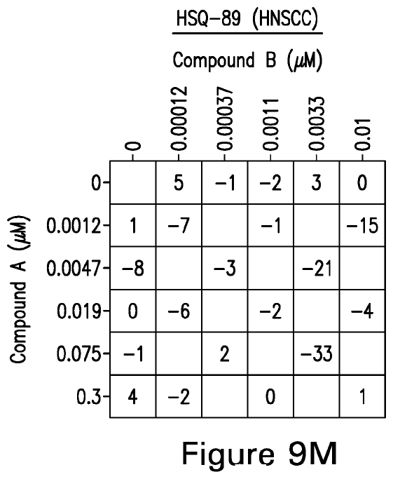

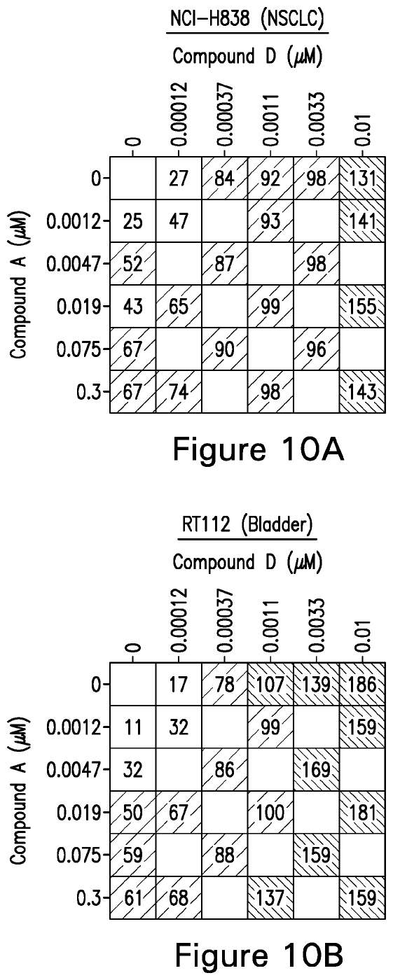

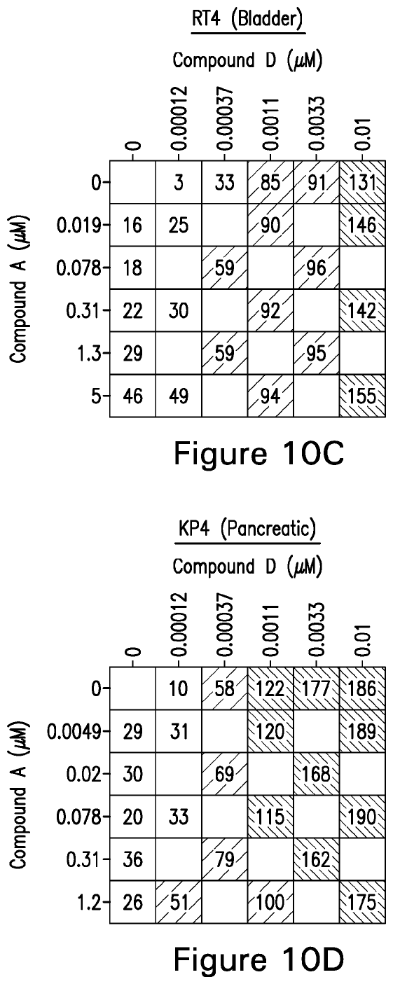

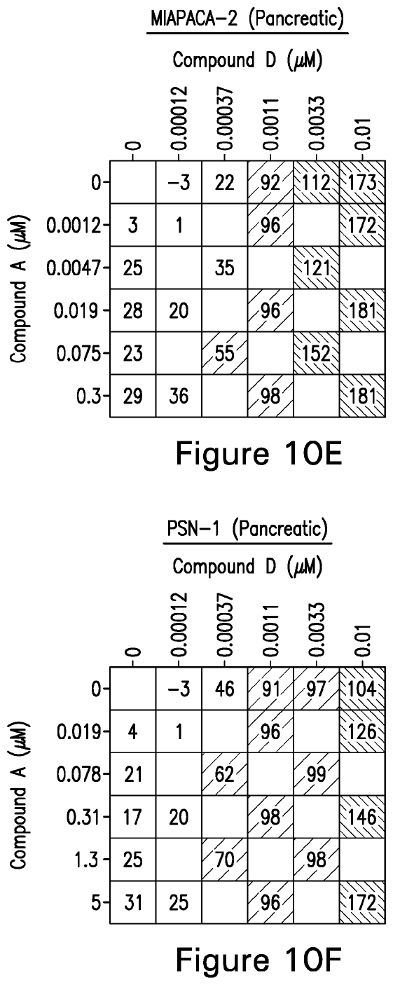

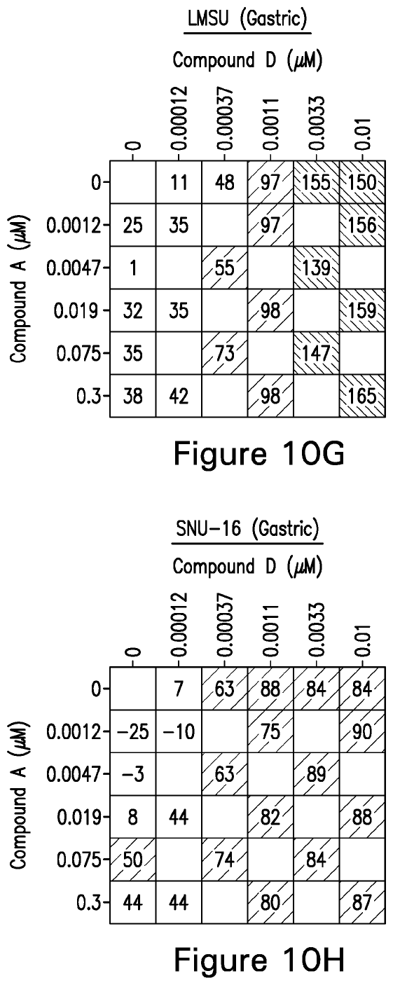

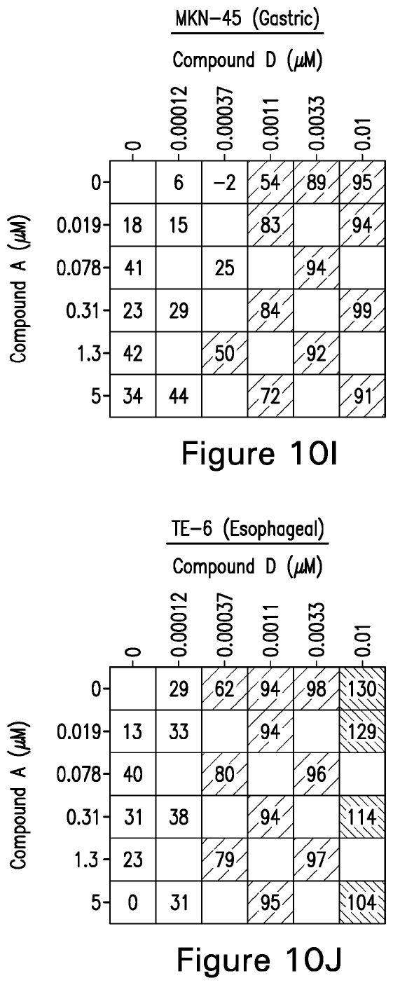

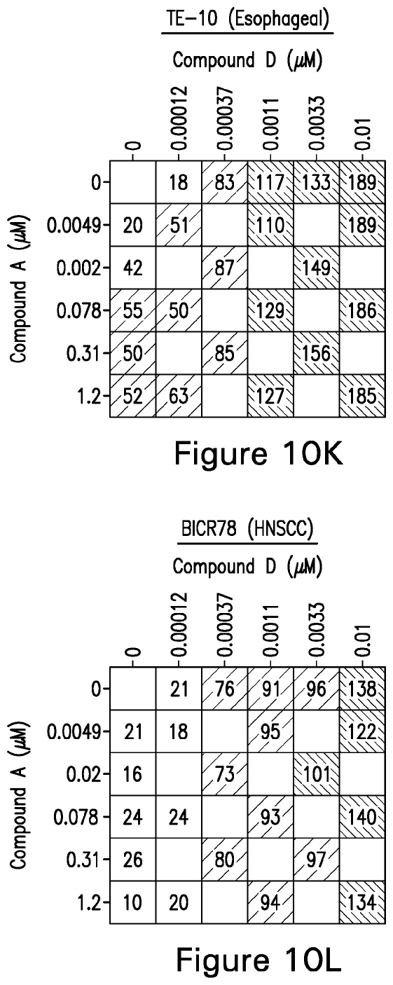

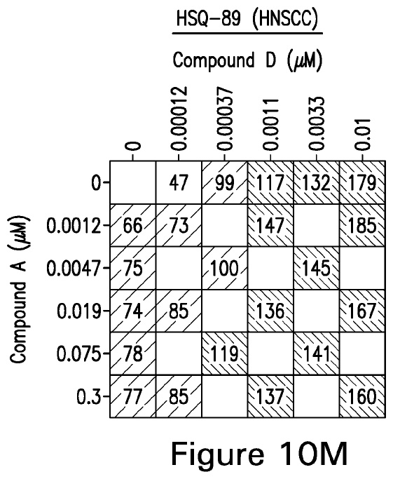

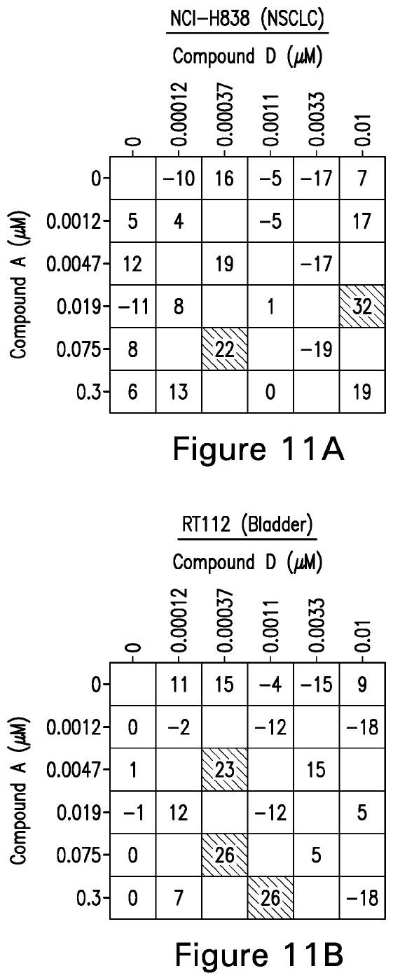

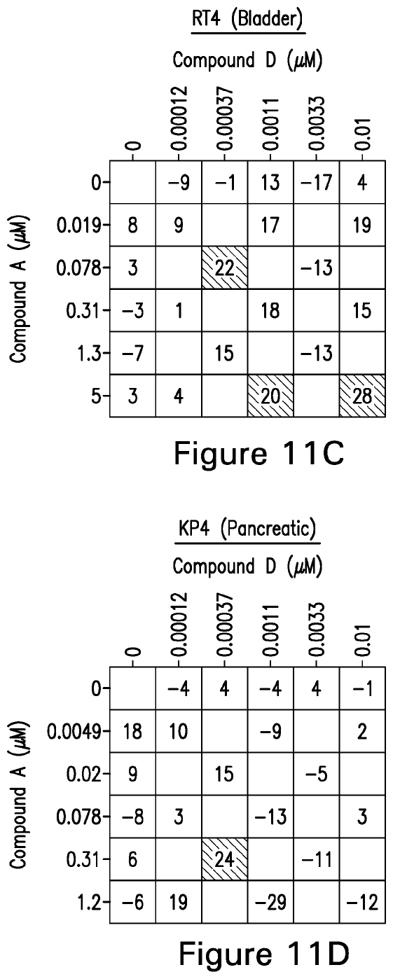

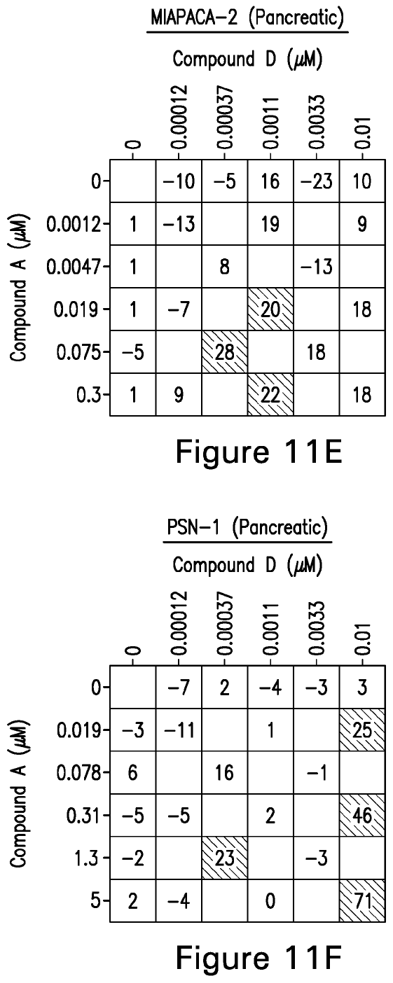

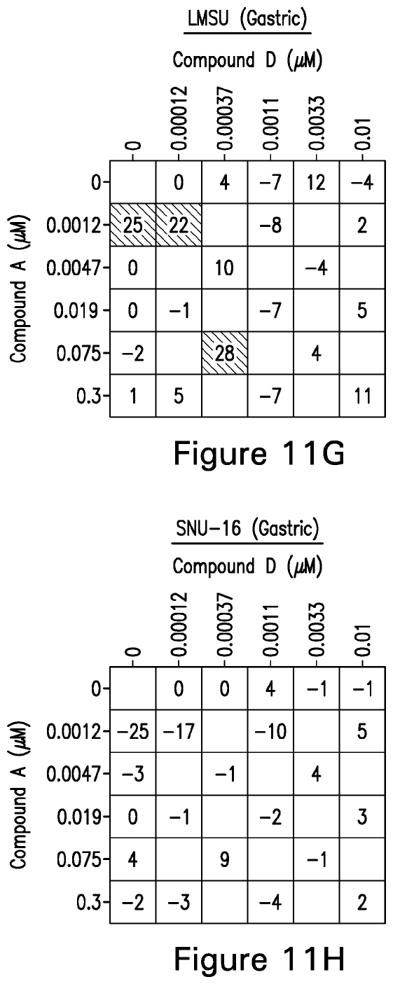

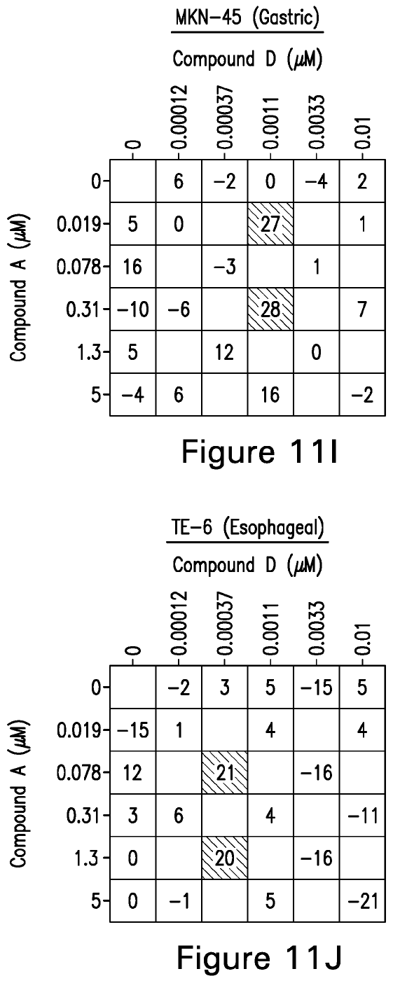

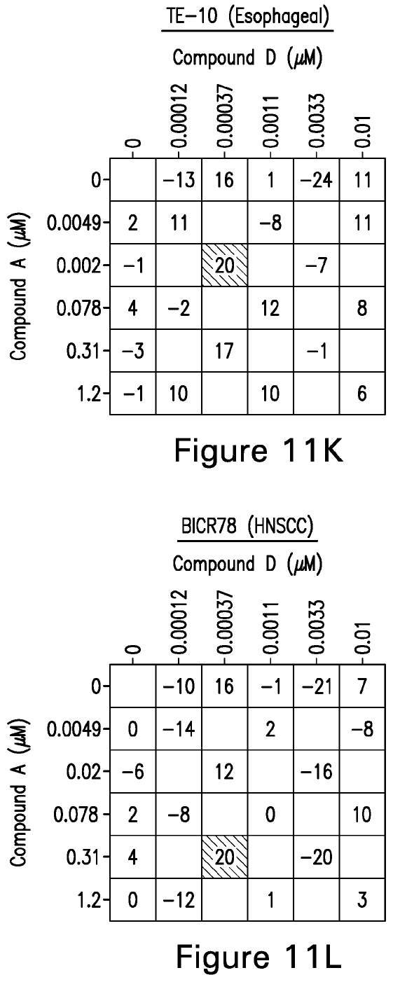

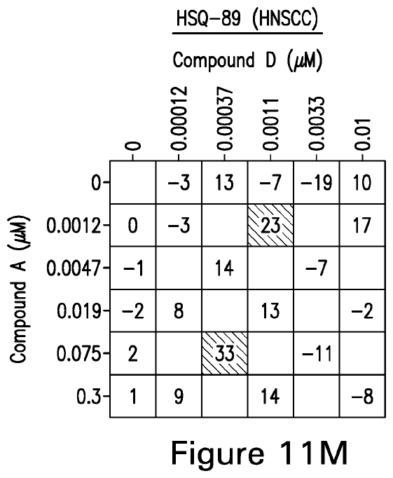

Synergy is measured at all tested concentrations of Compound A and Compound B, as well as Compound A and Compound D, using the Loewe additivity model for each cell line. Scores >20 are highlighted in dark gray and are considered synergistic in FIGS. 9A-M, 10A-M, and 11A-M.

Taken together, the synergy of Compound A and either Compound B or D across multiple MTAP-deficient models of various indications, respectively, suggests the potential benefit of these combinations in MTAP-deleted cancer patients. Importantly, the synergistic activity of Compound A and B in pancreatic cancer cell lines and Compound A and D in bladder cancer cell lines highlight the potential of these tumor types to benefit from these combination therapies.

TABLE 9
Synergy scores for the combination of Compound A and either
Compound B or D in 13 MTAP-deficient cell lines.

| | | Cmpd A × Cmpd B | Cmpd A × Cmpd D |
|---|---|---|---|
| Cell Line | Indication | Synergy Score | Synergy Score |
| NCI-H838 | NSCLC | 10.5 | 4.53 |
| RT112 | Bladder | 2.32 | 3.88 |
| RT4 | Bladder | 3.96 | 4.97 |
| KP4 | Pancreatic | 3.98 | 1.56 |
| MIAPACA-2 | Pancreatic | 3.69 | 5.56 |
| PSN-1 | Pancreatic | 4.7 | 7.34 |
| LMSU | Gastric | 3.71 | 2.05 |
| SNU-16 | Gastric | 1.06 | 0.54 |
| MKN-45 | Gastric | 1.56 | 2.18 |
| TE-6 | Esophageal | 2.57 | 1.53 |
| TE-10 | Esophageal | 2.96 | 3.45 |
| BICR78 | HNSCC | 1.14 | 1.36 |
| HSQ-89 | HNSCC | 0.115 | 4.93 |

Scores >3.0 are considered synergistic.

### Example 7: MAT2A Inhibition and Docetaxel Combination in 3 In Vitro MTAP-Deficient Models

### Materials and Methods

The HCT116 isogenic pair, stably transduced with Incucyte Nuclight Red Lentivirus Reagent, and an endogenous MTAP^{-/-} pancreatic cancer cell line, KP4, were used to assess the combinatory effect of a MAT2A inhibitor (Compound A) and Docetaxel (Compound D) in vitro. Cells were seeded at a density of 1,000 cells/well in a 96-well plate, and 24 hrs later, treated with a 6-point, 5-fold titration series of Compound A, starting from the highest concentration of 10 μM, and a 7-point, 5-fold titration series of Compound D, starting from the highest concentration of 0.47 μM in a 7×8 double matrix, using a TECAN liquid dispenser. After 5-6 cell population doublings (4 days for HCT116 isogenic pair and 5 days for KP4 cell line), cells were imaged with an IncuCyte S3 Live-Cell Analysis System for nuclear count determination. Only the KP4 cells were incubated with 5 μM of Vybrant DyeCycle Green for 90 minutes prior to being imaged. Each data point were run in technical triplicate.

The nuclear counts obtained at the terminal timepoint were normalized to the average nuclear counts of the DMSO (vehicle control)-treated cells, as a percent of the control (POC), where POC=(X/average nuclear cell counts of DMSO control)×100. Dose response curves and absolute or relative IC₅₀ calculations for single drug activity and drug combination synergy were generated using the Combenefit software, and synergy was evaluated using HSA, Bliss, and Loewe models.

### Results

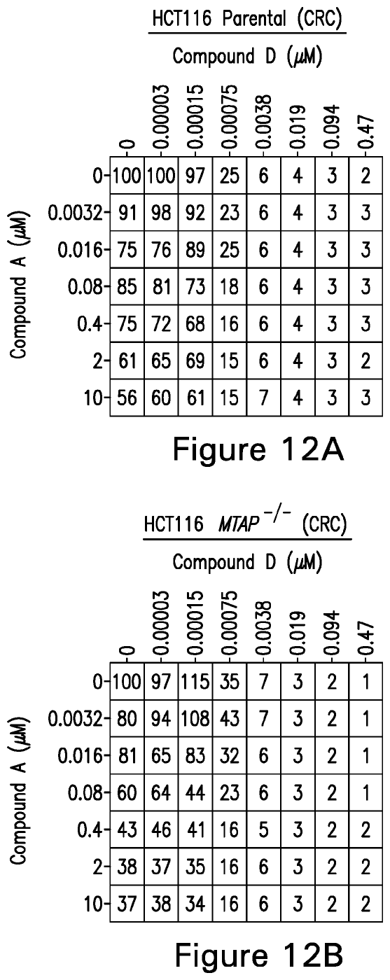

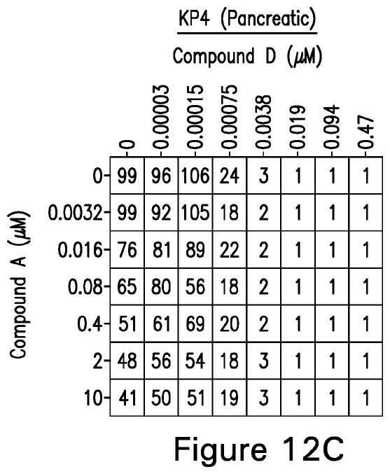

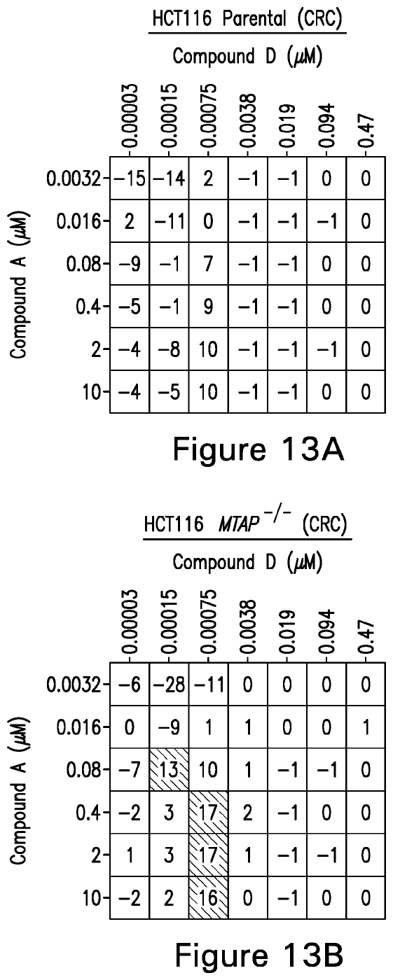

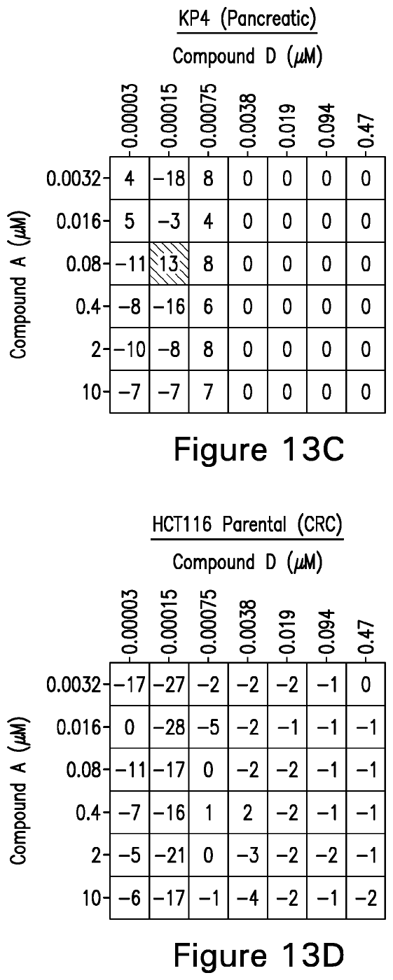

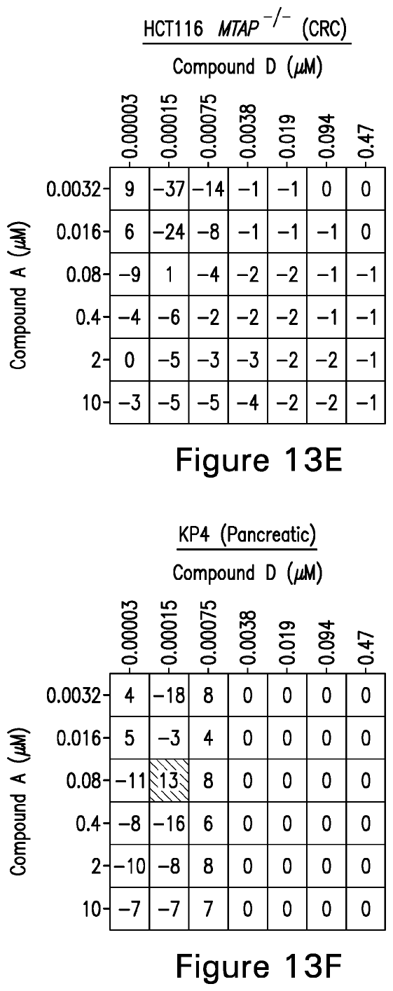

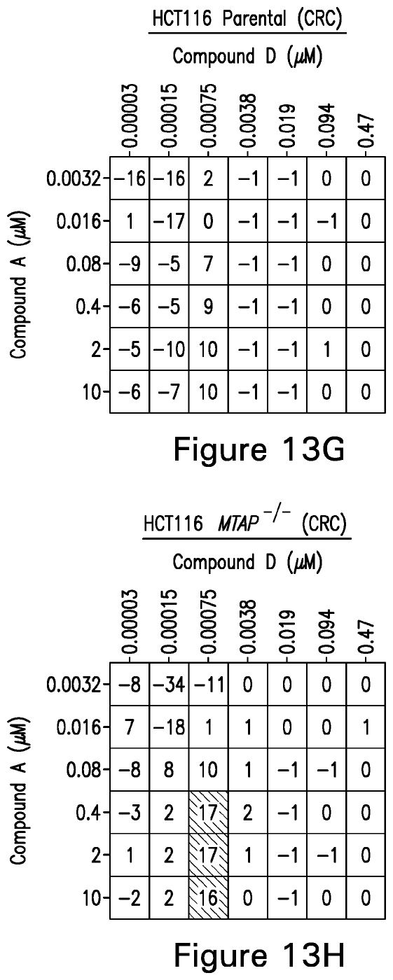

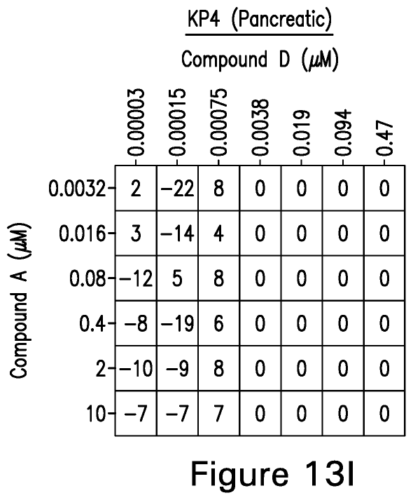

The combination of Compounds A and D demonstrated little to no synergy in the HCT116 isogenic pair and KP4 cell lines across all 3 models of synergy (FIGS. 12A-C and 13A-I). This could be due high sensitivity of all 3 models to Compound D as a single agent and the steep dose response curve that may limit the assay window of observing synergy. This result may also be model-dependent and not indicative of the combinatory activity of Compounds A and D in other MTAP-deficient cell lines. In FIG. 12, relative nuclear counts are presented as a percent of the DMSO control-treated cells for each cell line in a 7×8 dose matrix. In FIG. 13A-E, Synergy is measured at all tested concentrations of Compound A and Compound D using the HSA, Bliss, or Loewe model from the Combenefit software. Scores >10 are highlighted in dark gray and are considered synergistic.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

Other embodiments are within the following claims.

1. A method of treating cancer in a subject in need thereof, the method comprising administering to the subject a therapeutically effective amount of a methionine adenosyltransferase II alpha (MAT2A) inhibitor and administering to the subject a therapeutically effective amount of a taxane, wherein the MAT2A inhibitor is a compound of Formula IIIa or IIId: 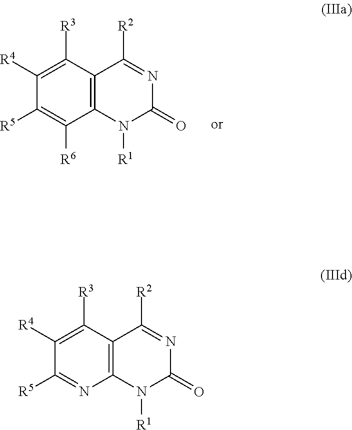
or a pharmaceutically acceptable salt thereof;
wherein
R³ is hydrogen;
R⁵ is alkyl, alkoxy, halo, haloalkyl, haloalkoxy, cycloalkyl, cyano, aminocarbonyl, heteroaryl, heterocyclyl;
R⁴ and R⁶ are independently hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d};
R² is alkyl, haloalkyl, hydroxyalkyl, alkoxyalkyl, aminoalkyl, aminocarbonylalkyl, aminosulfonylalkyl, or —NR⁹R¹⁰, wherein:
R⁹ is hydrogen or alkyl;
R¹⁰ is hydrogen; and
R^{d} is selected from alkyl, haloalkyl, haloalkoxy, alkoxy, hydroxy, alkylsulfonyl, halo, and cyano.

2. The method of claim 1, wherein the MAT2A inhibitor is a compound of Formula IIId, or a pharmaceutically acceptable salt thereof.

3. The method of claim 1 or 2, wherein R⁵ is chloro, methyl, ethyl, trifluoromethyl, 1,1-difluoroethyl, or cyclopropyl.

4. The method of any one of claims 1-3, wherein
R¹ is R⁷;
R⁷ is phenyl which is unsubstituted or substituted with R^{f}; and
R^{f} is selected from alkyl, haloalkyl, haloalkoxy, alkoxy, hydroxy, halo, and cyano.

5. The method of any one of claims 1-4, wherein the MAT2A inhibitor is selected from the group consisting of a compound from Table 7, or a pharmaceutically acceptable salt thereof.

6. The method of any one of claims 1-5, wherein the MAT2A inhibitor is Compound A: 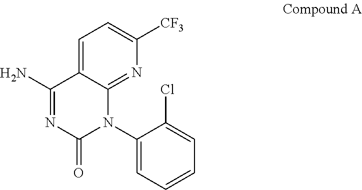 [SMILES: Nc1nc(=O)n(-c2ccccc2Cl)c2nc(C(F)(F)F)ccc12]
or a pharmaceutically acceptable salt thereof.

7. The method of any one of claims 1-5, wherein the MAT2A inhibitor is Compound A1: 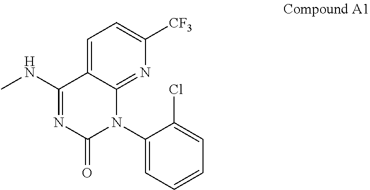 [SMILES: CNc1nc(=O)n(-c2ccccc2Cl)c2nc(C(F)(F)F)ccc12]
or a pharmaceutically acceptable salt thereof.

8. The method of any one of claims 1-7, wherein the taxane is selected from the group consisting of 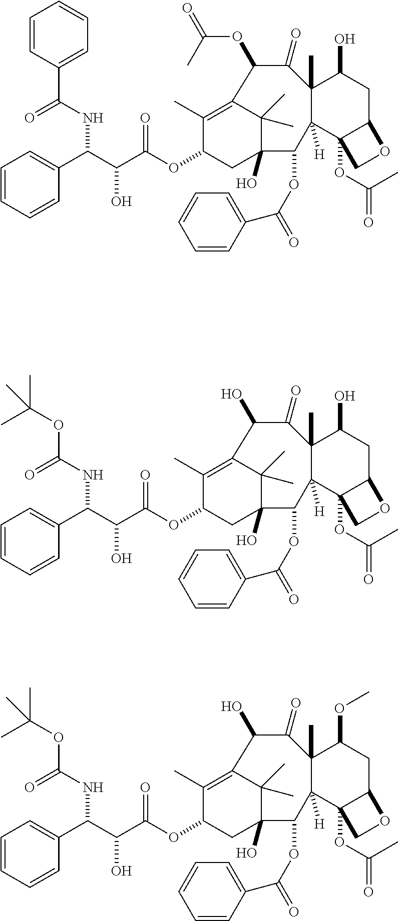
or a pharmaceutically acceptable salt or hydrate thereof.

9. The method of any one of claims 1-8, wherein the taxane is Compound B: 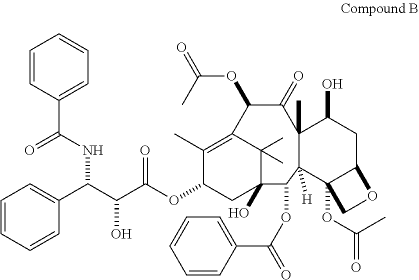
or a pharmaceutically acceptable salt thereof.

10. The method of any one of claims 1-7, wherein the taxane is protein-bound paclitaxel (Compound C).

11. The method of any one of claims 1-8, wherein the taxane is Compound D: 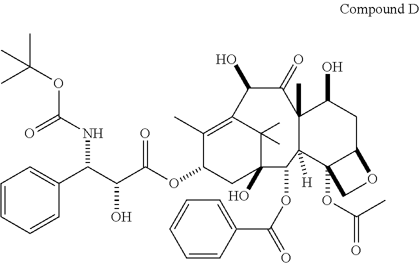
or a pharmaceutically acceptable salt thereof.

12. A method of treating cancer in a subject in need thereof, the method comprising administering to the subject a therapeutically effective amount of Compound A 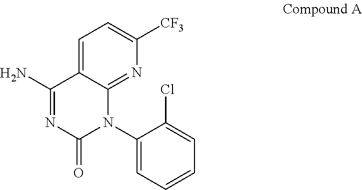 [SMILES: Nc1nc(=O)n(-c2ccccc2Cl)c2nc(C(F)(F)F)ccc12]
or a pharmaceutically acceptable salt thereof,
and administering to the subject a therapeutically effective amount of a taxane, or a pharmaceutically acceptable salt thereof.

13. A method of treating cancer in a subject in need thereof, the method comprising administering to the subject a therapeutically effective amount of Compound A1 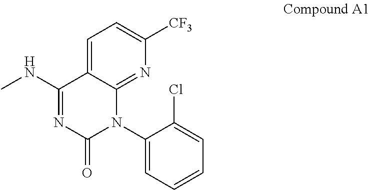 [SMILES: CNc1nc(=O)n(-c2ccccc2Cl)c2nc(C(F)(F)F)ccc12]
or a pharmaceutically acceptable salt thereof,
and administering to the subject a therapeutically effective amount of a taxane, or a pharmaceutically acceptable salt thereof.

14. The method of claim 12 or 13, wherein the taxane is Compound B: 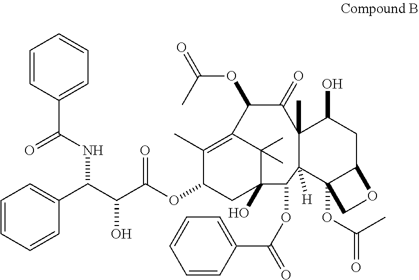
or a pharmaceutically acceptable salt thereof.

15. The method of claim 12 or 13, wherein the taxane is protein-bound paclitaxel (Compound C).

16. The method of claim 12 or 13, wherein the taxane is Compound D: 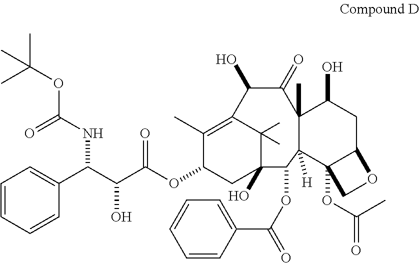
or a pharmaceutically acceptable salt thereof.

17. The method of any one of claims 1-16, wherein the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, esophagogastric cancer, malignant peripheral nerve sheath tumor, and mesothelioma.

18. The method of any of claims 1-16, wherein the cancer is a solid tumor.

19. The method of any of claims 1-16, wherein the cancer is a solid malignant tumor.

20. The method of claim 18 or 19, wherein the tumor has an MTAP gene deletion.

21. The method of any one of claims 1-20, wherein the MAT2A inhibitor and the taxane are in separate dosage forms.

22. The method of any one of claims 1-20, wherein the MAT2A inhibitor and the taxane are in the same dosage form.

23. A combination product comprising a methionine adenosyltransferase II alpha (MAT2A) inhibitor, or a pharmaceutically acceptable salt thereof, and a taxane, or a pharmaceutically acceptable salt thereof.

24. The combination product of claim 23, wherein the MAT2A inhibitor is a compound of Formula I and the taxane is selected from the group consisting of a compound B, compound C, and compound D, or a pharmaceutically acceptable salt thereof.

25. The combination product of claim 23 or 24, wherein the MAT2A inhibitor is compound A or A1, and the taxane is selected from the group consisting of a compound B, compound C, and compound D, or a pharmaceutically acceptable salt thereof.

26. A methionine adenosyltransferase II alpha (MAT2A) inhibitor for use in treating cancer, wherein the MAT2A inhibitor is to be administered simultaneously or sequentially with a taxane.

27. The MAT2A inhibitor for use of claim 26, wherein the MAT2A inhibitor is a compound of Formula IIIa or IIId: 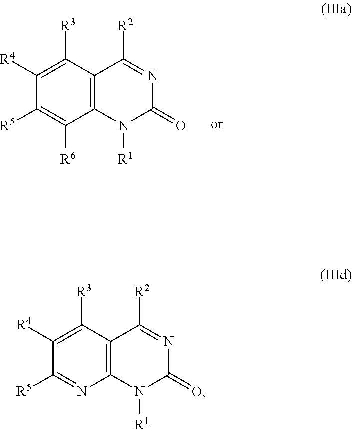
or a pharmaceutically acceptable salt thereof;
wherein
R³ is hydrogen;
R⁵ is alkyl, alkoxy, halo, haloalkyl, haloalkoxy, cycloalkyl, cyano, aminocarbonyl, heteroaryl, heterocyclyl;
R⁴ and R⁶ are independently hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d};
R² is alkyl, haloalkyl, hydroxyalkyl, alkoxyalkyl, aminoalkyl, aminocarbonylalkyl, aminosulfonylalkyl, or —NR⁹R¹⁰, wherein:
R⁹ is hydrogen or alkyl;
R¹⁰ is hydrogen; and
R^{d} is selected from alkyl, haloalkyl, haloalkoxy, alkoxy, hydroxy, alkylsulfonyl, halo, and cyano; and the taxane is selected from the group consisting of compound B, compound C, and compound D, or a pharmaceutically acceptable salt thereof.

28. The use of claim 27, wherein the MAT2A inhibitor is compound A or compound A1.

29. Use of a methionine adenosyltransferase II alpha (MAT2A) inhibitor in the manufacture of a medicament for treating cancer, wherein the MAT2A inhibitor is to be administered simultaneously or sequentially with a taxane.

30. The use of claim 29, wherein the MAT2A inhibitor is a compound of Formula IIIa or IIId: 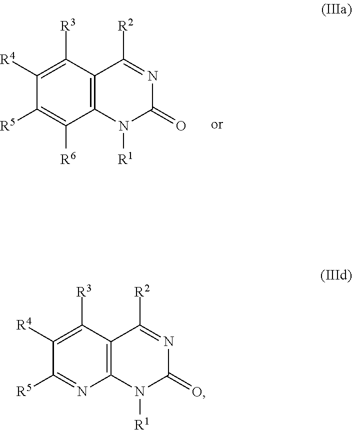
or a pharmaceutically acceptable salt thereof;
wherein
R³ is hydrogen;
R⁵ is alkyl, alkoxy, halo, haloalkyl, haloalkoxy, cycloalkyl, cyano, aminocarbonyl, heteroaryl, heterocyclyl;
R⁴ and R⁶ are independently hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d};
R² is alkyl, haloalkyl, hydroxyalkyl, alkoxyalkyl, aminoalkyl, aminocarbonylalkyl, aminosulfonylalkyl, or —NR⁹R¹⁰, wherein:
R⁹ is hydrogen or alkyl;
R¹⁰ is hydrogen; and
R^{d} is selected from alkyl, haloalkyl, haloalkoxy, alkoxy, hydroxy, alkylsulfonyl, halo, and cyano; and
the taxane is selected from the group consisting of compound B, compound C, and compound D, or a pharmaceutically acceptable salt thereof.

31. The use of claim 30, wherein the MAT2A inhibitor is compound A or compound A1.
